(19) United States
(12) Patent Application Publication
(10) Pub. No.: US 2007/0250941 A1
(43) Pub. Date: Oct. 25, 2007

(54) VEGF Receptor Conditional Knockout Animals and Methods of Use

(75) Inventors: Megan Baldwin, San Francisco, CA (US); Hans-Peter Gerber, Bellevue, WA (US); Napoleone Ferrara, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/691,469

(22) Filed: Mar. 26, 2007

Related U.S. Application Data
(60) Provisional application No. 60/786,223, filed on Mar. 27, 2006.

(51) Int. Cl. A01K 67/027; C12N 5/06; C07K 14/71

(52) U.S. Cl. 800/14; 800/18; 435/325; 435/354; 435/320.1; 530/350

(57) ABSTRACT

Transgenic non-human animals and methods of producing non-human transgenic animals comprising within their genome a conditional targeted mutation in a VEGFR, e.g., VEGFR1 or VEGFR2, gene are provided along with methods and vectors used to produce such animals or cells.

### RELATED APPLICATION

This application claims priority to and the benefit of U.S. Provisional Application Ser. No. 60/786,223, filed Mar. 27, 2006 specification of which is incorporated herein in its entirety.

### FIELD OF THE INVENTION

The invention relates to transgenic animals with conditional knockouts to VEGF receptors, along with vectors used in and methods of producing such animals. The animals can be used for analyzing VEGFR function and screening for agents to modulate VEGFR activities.

### BACKGROUND OF THE INVENTION

Vascular endothelial growth factor receptor (VEGFR)-1 and VEGFR-2, also known as fms-like tyrosine kinase (Flt-1) and fetal liver kinase 1 (Flk-1)/kinase insert domain receptor (KDR) respectively, are receptor tyrosine kinases that bind with high affinities to members of the VEGF family. For example, vascular endothelial growth factor (VEGF-A), one member of the VEGF family, is a secreted growth factor produced by a variety of cell types, which mediates its effector functions by binding and activation of VEGFR-1 and VEGFR-2. VEGF-A regulates a variety of vascular functions, including endothelial cell differentiation and survival, endothelium-dependent vasodilatation, microvascular hyperpermeability and interstitial matrix remodeling. See, e.g., Ferrara, N., et al. The biology of VEGF and its receptors. Nat Med 9:669-676(2003). More recently, VEGF receptor expression was identified on a variety of non-endothelial cells suggesting additional, non-vascular regulatory functions of this ligand/receptor system. See, e.g., Autiero, M., et al. Placental growth factor and its receptor, vascular endothelial growth factor receptor-1: novel targets for stimulation of ischemic tissue revascularization and inhibition of angiogenic and inflammatory disorders. J Thromb Haemost 1: 1356-1370(2003).

In vitro and in vivo studies and characterizations of vascular endothelial growth factor receptors (VEGFRs) can provide valuable identification and discovery of therapeutics and/or treatments useful in the prevention, amelioration or correction of diseases or dysfunctions associated with VEGFRs activity and/or expression. Conventional gene-targeting strategies demonstrated the essential roles of VEGFR-1 and VEGFR-2 during early embryonic development, as mouse embryos that are null for either receptor die between days 8.5 and 9.5 due to profound defects in angiogenesis and formation of a functional cardiovascular system. See, e.g., Carmeliet, P. et al., Abnormal Blood Vessel Development and Lethality in Embryos Lacking a Single VEGF Allele, Nature 380:435-439 (1996); Shalaby, F., et al., Failure of Blood-Island Formation and Vasculogenesis in Flk-1 Deficient Mice, Nature 376:62-66 (1995); Fong, G. H., et al., Role of the Flt-1 receptor tyrosine kinase in regulating the assembly of vascular endothelium, Nature 376:66-70 (1995); and, Fong, G. H., Increased Hemangioblast Commitment, not vascular disorganization, is the primary defect in Flt-1 knock-out mice, Development. 126:3015-3025 (1999). However, due to the early embryonic lethality of VEGFR-1 and VEGFR-2 null mice, the role of these receptors during early postnatal life and in the adult has remained unclear. This has precluded analysis of function of these receptors in later development. There is a need to discover and understand the biological functions of VEGF receptors and their ligands in various organs and cells types of the body during various stages of development and disease. The invention addresses these and other needs, as will be apparent upon review of the following disclosure.

### SUMMARY OF THE INVENTION

VEGFR transgenic knockout animals are described and uses of these transgenic animals are provided. VEGFR transgenic conditional knockout animals are animals in which a VEGFR gene is conditionally inactivated. In certain embodiments, a VEGFR gene is conditionally inactivated in specific tissues or during a specific stage in development or age. In certain embodiments of the invention, the VEGFR is VEGFR-1 or VEGFR-2. The transgenic non-human animal can be homozygous or heterozygous for the disruption of a desired gene, e.g., encoding for a VEGFR-1, VEGFR-2 or other receptor and/or ligand. The invention also provides a transgenic non-human animal comprising, e.g., a VEGFR-1-Cre transgenic non-human animal, VEGFR-2-Cre transgenic non-human animal, a conditional VEGFR-1-loxP transgenic non-human animal, or a conditional a VEGFR-2 loxP transgenic non-human animal. The transgenic non-human animal can be any animal, e.g., a rodent, a rat, a mouse, a rabbit, a monkey, a guinea pig, a dog, a sheep, a horse, a dog, a cow, a cat, etc. The invention also provides an isolated cell derived from a transgenic non-human animal whose genome comprises a disruption and/or conditional disruption of at least one desired gene. In certain embodiments of the invention, the at least one desired gene encodes for a VEGFR. In certain embodiments, the isolated cell comprises an embryonic stem cell, e.g., from a rodent, a rat, a mouse, a rabbit, a monkey, a guinea pig, a dog, a sheep, a horse, a dog, a cow, a cat, etc.

In certain embodiments of the invention, there can be more than one gene inactivated and/or conditionally inactivated, e.g., inactivating VEGFR-1 and VEGFR-2, inactivating VEGFR-1 and VEGF, inactivating VEGFR-2 and VEGF, or inactivating VEGFR-1 and/or VEGFR-2 along with another receptor(s) (e.g., VEGFR-3 (Flt-4), another receptor that binds VEGF, a tie receptor, etc.), and/or ligand(s) (e.g., VEGF-C, VEGF-B, P1GF, VEGF-D, etc.), in a transgenic non-human animal. In one embodiment of the invention, a transgenic non-human animal of the invention comprises, e.g., a VEGFR-1-Cre; VEGF-loxP transgenic non-human animal. In one embodiment of the invention, a transgenic non-human animal of the invention comprises, e.g., a VEGFR-2-Cre; VEGF-loxP transgenic non-human animal.

The invention also provides vectors and methods for producing transgenic non-human animals comprising within their genome a targeted mutation or conditional targeted mutation within a VEGFR gene, e.g., a VEGFR-1 gene or a VEGFR-2 gene. In certain embodiments of the invention, a vector construct is provided that comprises the whole or a fragment of the genomic sequence of a VEGFR gene, where the genomic sequence further comprises a selection marker, e.g., a positive selection marker, and where at least part of an exon of VEGFR gene is framed by recognition sites for a recombinase. In certain embodiments of the invention, a vector construct is provided that comprises the whole or a fragment of the genomic sequence of a VEGF gene, where the genomic sequence further comprises a selection marker, e.g., a positive selection marker, and where at least part of an exon of VEGF gene is framed by recognition sites for a recombinase In certain embodiments of the invention, a vector construct is provided that comprises the whole or a fragment of the genomic sequence of a VEGFR gene, where the genomic sequence further comprises a nucleic acid sequence encoding a recombinase.

Examples of positive selection markers include, but are not limited to, e.g., a neomycin resistance gene (neo), a hygromycin resistance gene, etc. In one embodiment, the positive selection marker is a neomycin resistance gene. In certain embodiments of the invention, the genomic sequence further comprises a negative selection marker. Examples of negative selection markers include, but are not limited to, e.g., a diphtheria toxin gene, an HSV-thymidine kinase gene (HSV-TK), etc.

When the genomic sequence of a VEGFR gene comprises at least part of an exon of a VEGFR gene that is framed by recognition sites for a recombinase, upon the initialization of the conditional mutation, the at least part of an exon of a VEGFR gene can then be deleted with the help of a recombinase. The recognition sites for a recombinase include, but are not limited to, e.g., frt sites for a flp recombinase and lox sites for a cre recombinase. Typically, the vector comprises enough of the genomic sequence of a VEGFR gene necessary for homologous recombination as well as part of the genomic sequence comprising part of an exon of a VEGFR gene, or one exon or more exons of a VEGFR gene framed by recognition sites of a recombinase. In certain embodiments of the invention, the genomic sequence encodes part of the amino acid sequence of a VEGFR protein involved in its function and activity, which is framed by recognition sites for a recombinase. In certain embodiments of the invention, the genomic sequence of the VEGFR gene comprises at least exon 1. In one embodiment, exon 1 of a VEGFR gene are framed by recognition sites for a recombinase. In certain embodiments of the invention, the genomic sequence of the vector construct may comprise all exons of a VEGFR gene. In one embodiment, VEGFR gene is a murine sequence.

In certain embodiments of the invention, a transgenic non-human animal is provided in which a VEGFR gene is inactivated by expression of the cre recombinase in cells with the genomic sequence of a VEGFR gene, where the genomic sequence comprises at least part of an exon of a VEGFR gene that is framed by recognition sites for a recombinase. In one embodiment of the invention, the VEGFR gene is functionally inactivated by the recombinase mediated generation of a deletion within a VEGFR gene. In certain embodiments of the invention, there is reduced or no significant expression of the VEGFR gene in cells of the transgenic non-human animal. In certain embodiments of the invention, the functionally reduced or inactivated VEGFR gene expression results in reduced or no functional VEGFR protein compared to conditional transgenic non-human animal not induced or a wild-type control animal. These animals are useful for studying and identifying compositions that modulate VEGFR.

The invention also provides methods of producing a transgenic non-human animal, whose one or both alleles of a VEGFR or VEGF gene are mutated and/or truncated in a way that when triggered by an inducer less than the normal amount or no active VEGFR or VEGF protein is expressed compared to a non-induced control or wild-type control. For example, methods include introducing a vector as described herein into an embryonic stem cell of a non-human animal; and generating a heterozygous and/or homozygous transgenic animal from the embryonic stem cell, thereby producing the transgenic conditional targeted mutation non-human animal. In a further embodiment, the above-described method further comprises crossbreeding the transgenic conditional targeted mutation non-human animal with an animal transgenic for the recombinase recognizing the recognition sites framing the positive selection marker within the genomic sequence of a VEGFR or VEGF gene, where the genomic sequence comprises at least part of an exon of a VEGFR gene or of an exon of a VEGF gene. In one embodiment, the genomic sequence is at least part of exon 1 of a VEGFR gene. In one embodiment, the genomic sequence is at least part of exon 3 of a VEGF gene. Based on the specific conditional targeting construct a VEGFR-deficient or a VEGF and VEGFR deficient animal may be generated. These animals may be analyzed genetically, histologically, electrophysiologically, and behaviorally. In one embodiment, the above-described method additionally comprises further crossbreeding the transgenic conditional targeted mutation non-human animal with an animal transgenic for the recombinase recognizing the recognition sites framing the positive selection marker within the genomic sequence of a VEGFR, where the genomic sequence comprises at least part of an exon of a VEGFR gene.

In some embodiments of the invention, the animal transgenic for the recombinase expresses it in a tissue-specific manner. In certain embodiments of the invention, a mouse line with an inducible promoter is used. For example, a Mx1 promoter can be used which then can be activated by an inducer. In certain embodiments of the invention, an inducer includes, but is not limited to, an agent (such as interferon-alpha, interferon-beta, a synthetic double-stranded RNA (poly inosinic-polycytidylic acid, etc.) or cell or animal that produces expression of the cre recombinase, e.g., by cross-breeding with an animal transgenic for the recombinase. In certain embodiments of the invention, additional marker may be included in the cell or non-human animal. The marker, e.g., a histological marker, can be activated by recombinase indicating Cre activity. Markers include, e.g., beta-galactosidase, enhanced fluorescent proteins of green, yellow or cyan types, lacZ gene expression, alkaline phosphatase expression, etc.

Methods of identifying an agent that alters the function of a VEGFR and/or VEGF in an animal lacking a functional VEGFR and/or VEGF gene product as compared with the animal expressing a functional VEGFR and/or VEGF gene product are provided. For example, a method comprises comparing at least one response of a transgenic non-human animal having a targeted conditional mutation in a VEGFR and/or VEGF gene or cells of the transgenic non-human animal with a non-human animal expressing a functional VEGFR and/or VEGF gene product; contacting the transgenic non-human animal an a test agent; and, identifying the agent (e.g., a VEGFR modulator or a VEGF modulator). Typically, the agent alters the at least one response compared with the non-human animal, thus altering the function of the VEGFR and/or VEGF.

Methods of identifying a phenotype associated with a conditional disruption of a gene which encodes for a VEGFR-1, VEGFR-2 and/or VEGF are also provided. A phenotype resulting from the gene disruption is identified as the physiological characteristic of the transgenic conditional non-human animal that differs from the physiological characteristic of the wild type animal, when the condition is initiated. Methods for identifying an agent that modulates a phenotype associated with a conditional disruption of a gene that encodes for a VEGFR-1, VEGFR-2, and/or VEGF are also provided. For example, a method comprises comparing at least one phenotype of a transgenic non-human animal having a targeted conditional mutation in a VEGFR and/or VEGF gene or cells of the transgenic non-human animal with a non-human animal expressing a functional VEGFR and/or VEGF gene product; contacting the transgenic non-human animal with a test agent; and, identifying the agent (e.g., a VEGFR modulator or a VEGF modulator). The agent alters at least one phenotype compared with the non-human animal, which identifies an agent that alters the function of the VEGFR and/or VEGF.

Methods of identifying an agent (e.g., a VEGFR modulator or a VEGF modulator) that modulates a disorder associated with a conditional disruption of the gene which encodes for a VEGFR-1, VEGFR-2 and/or VEGF are also provided. Methods of identifying an agent that ameliorates a disorder associated with a conditional disruption in the gene which encodes for a VEGFR-1, VEGFR-2 or VEGF are provided. For example, a method includes (a) administering a test agent to a non human transgenic animal comprising the conditional disruption in a VEGFR-1, VEGFR-2 and/or VEGF gene; and (b) determining whether the test agent modulates or ameliorates the disorder associated with the gene disruption in the non human transgenic conditional animal. If the test agent modulates or ameliorates the disorder, it is an agent (e.g., a VEGFR modulator or a VEGF modulator).

An agent identified by any of above methods is also included in the invention. In one embodiment, the agent comprises an agonist. In another embodiment, the agent comprises an antagonist of a VEGFR-1, VEGFR-2 and/or VEGF. Agents that are therapeutic agents are also included in the invention along with a pharmaceutical composition including the therapeutic agent.

### BRIEF DESCRIPTION OF THE FIGURES

FIG. 1, Panels a-f, illustrates In Vitro Analysis of VEGF-A and Flt1-Deficient Mesangial Cells:

(a) Gene-targeting strategy to create Flt1-loxP mice. The targeting vector was designed to introduce a PGK-Neo cassette flanked by 2 loxP sites (LoxP1 and LoxP2) upstream of the first exon containing the translation initiation codon (ATG) of the Flt1 gene, and to introduce a third loxP site (LoxP3) 3′ to the first exon. Following Cre-recombinase expression, embryonic stem (ES) cell clones that had undergone recombination between LoxP1 and LoxP2 were selected and used to generate Flt1-loxP mice. The positions of the PCR-amplified genomic DNA probes (5′ Pr, 3′ Pr) used to screen for targeting events and recombination by Southern blotting are shown. The position of restriction enzyme sites used in this screening and the size of the regions of the targeting vector (in kilobases) are as indicated. E: EcoRI; H: HindIII; K: KpnI; kb: kilobases. (b) Southern blot analysis of genomic DNA extracted from WT and targeted (Targ.) ES cell clones, and from Flt1^{(loxP/loxP)} mesangial cells (Mes.) infected with adenovirus encoding LacZ (LZ) or Cre-recombinase (Cre) genes. Genomic DNA from the respective cells was digested with either EcoRI or both HindIII and KpnI to analyse for targeting events and loxP recombination at either the 5′ end or 3′ end of the targeted regions of the Flt1 gene respectively. The sizes of the expected fragments detected in each of the lanes is indicated (in kilobases) to the left and right of each panel respectively. (c) VEGF-A expression in mesangial cells infected with adenovirus. Mesangial cells were isolated from WT and VEGF^{(loxP/loxP)} mice and infected with adenovirus encoding LacZ (Ad-LacZ) or Cre-recombinase (Ad-Cre). Total RNA was isolated and subjected to quantitative real-time PCR for the analysis of VEGF-A expression. Results are expressed as relative RNA units (RRU) following standardization to GAPDH, and standard curves for each primer/probe set were generated using total kidney RNA from WT mice. (d) Flt1 expression in mesangial cells infected with adenovirus. Mesangial cells from WT and Flt1^{(loxP/loxP)} mice were isolated and infected with Ad-LacZ and Ad-Cre. RNA was isolated and analysed for Flt1 expression by quantitative RT-PCR. Results are expressed as described in C. (e) Survival of VEGF-A and Flt1-deficient mesangial cells in vitro. The cell count ratio between Ad-LacZ and Ad-Cre treatments was calculated and normalized as a percentage of the value obtained for the WT cells. Both VEGF-A and Flt1-deficient mesangial cells exhibited significantly reduced survival in vitro compared with WT mesangial cells. Statistically significant differences in survival compared to WT cells are noted by asterisks: *p value<0.05; **p value<0.01. Flt1-deficient and VEGF-deficient mesangial cells also exhibit significantly different survival in vitro, p<0.05. (f) Survival of Ad-LacZ infected mesangial cells cultured in the presence of a neutralizing VEGF antibody (α-VEGF) or control antibody (control IgG). The decrease in mesangial cell survival evident when either VEGF-A or Flt1 is ablated genetically was not recapitulated by culturing mesangial cells in the presence of α-VEGF.

FIG. 2, panels a and b schematically illustrate the generation of a conditional knockout of VEGFR-1 and VEGFR-2 (Panel a) and illustrate the identification of the embroyic stem cell clones in which the PGK neo cassette was removed but the two LoxP sites that flank exon 1 were maintained (Panel b).

FIG. 3 illustrates dosing of the conditional knockout VEGFR-1 and VEGFR-2 with IFN-alpha.

FIG. 4 illustrates VEGFR-1 expression in Mx1Cre, VEGFR-1-LoxP mice in bone marrow, liver spleen, kidney and thymus and VEGFR-2 expression in Mx1Cre, VEGFR-2-LoxP mice in bone marrow, liver spleen, kidney and thymus.

FIG. 5 illustrates B.U.N. and Creatin levels from VEGFR-1 conditional knockout animals (cKO) and VEGF-2 cKO.

FIG. 6, panels a-c, illustrate gene targeting and screening strategies to generate and analyse Flk1-LoxP mice. Panel a illustrates a gene-targeting strategy to create Flk1-LoxP mice. The targeting vector was designed to introduce a PGK-Neo cassette flanked by two loxP sites (LoxP1 and LoxP2) upstream of the first exon containing the translation initiation codon (ATG) of the Flk1 gene, and to introduce a third loxP site (LoxP3) 3′ to the first exon. Following Cre-recombinase expression, embryonic stem (ES) cell clones that had undergone recombination between LoxP1 and LoxP2 were selected and used to generate Flk1-loxP mice. The positions of the PCR-amplified genomic DNA probes (5′ Probe, 3′ Probe) used to screen for targeting events and recombination by Southern blotting are shown. The position of restriction enzyme sites used in this screening are as indicated (EcoRV; HindIII; and XbaI). Panel b illustrates a strategy to screen Flk1-LoxP ES cell clones and Flk1-LoxP mice by Southern Blot analysis. The expected size of the genomic DNA fragments to be detected by Southern Blot analysis using the 5′ Probe, and the 3′ Probe (positions indicated in FIG. 6, Panel A), in wild-type (WT) and targeted cells are indicated schematically. Panel c illustrates a strategy to screen Flk1-LoxP ES cell clones and Flk1-LoxP mice by PCR. The primer pairs used to screen the clones and Flk1-LoxP mice are indicated. The positions of the primer pairs relative to the targeting vector and genomic DNA are indicated in FIG. 6, panel a.

### DETAILED DESCRIPTION

Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “VEGF receptor” or “VEGFR” as used herein refers to a cellular receptor for VEGF, ordinarily a cell-surface receptor found on vascular endothelial cells, as well as fragments and variants thereof which retain the ability to bind VEGF (such as fragments or truncated forms of the extracellular domain). Some examples of VEGFR include the protein kinase receptors referred to in the literature as Flt-1 (also used interchangeably herein “VEGFR-1”) and KDR/Flk-1 (also used interchangeably herein “VEGFR-2”). See, e.g., DeVries et al. Science, 255:989 (1992); Shibuya et al. Oncogene, 5:519 (1990); Matthews et al. Proc. Nat. Acad. Sci., 88:9026 (1991); Terman et al. Oncogene, 6:1677 (1991); and Terman et al. Biochem. Biophys. Res. Commun., 187:1579 (1992). The Flt-1 (fms-like-tyrosine kinase) and KDR (kinase domain region) receptors bind VEGF with high affinity. Flk-1 (fetal liver kinase-1), the murine homolog of KDR, shares 85% sequence identity with human KDR. Ferrara Kidney Intl. 56:794-814 (1999). Both Flt-1 and KDR/Flk-1 have seven immunoglobulin (Ig)-like domains in the extracellular domain (ECD), a single transmembrane region and a consensus tyrosine kinase (TK) sequence, which is interrupted by a kinase-insert domain. Flt-1 has the highest affinity for rhVEGF₁₆₅, with a Kd of approximately 10 to 20 pM. KDR has a lower affinity for VEGF, with a Kd of approximately 75 to 125 pM. The nucleic acid sequences and amino acids sequences of a VEGFR are readily accessible and obtainable by one of skill in the art.

Other VEGF receptors include those that can be cross-link labeled with VEGF, or that can be co-immunoprecipitated with KDR or Flt-1. An additional VEGF receptor that binds VEGF₁₆₅ but not VEGF₁₂₁ has been identified. Soker et al Cell 92:735-45 (1998). The isoform-specific VEGF binding site is identical to human neuropilin-1, a receptor for the collapsin/semaphorin family that mediates neuronal cell guidance.

The terms “VEGF” and “VEGF-A” are used interchangeably to refer to the 165-amino acid vascular endothelial cell growth factor and related 121-, 145-, 183-, 189-, and 206-amino acid vascular endothelial cell growth factors, as described by Leung et al. Science, 246:1306 (1989), Houck et al. Mol. Endocrin., 5:1806 (1991), and, Robinson & Stringer, Journal of Cell Science, 144(5):853-865 (2001), together with the naturally occurring allelic and processed forms thereof. The term “VEGF” is also used to refer to fragments of the polypeptide, e.g., comprising amino acids 8 to 109 or 1 to 109 of the 165-amino acid human vascular endothelial cell growth factor. The amino acid positions for a “fragment” native VEGF are numbered as indicated in the native VEGF sequence. For example, amino acid position 17 in fragment native VEGF is also position 17 in native VEGF. The fragment native VEGF can have binding affinity for the KDR and/or Flt-1 receptors comparable to native VEGF.

The term “gene” refers to (a) a gene containing a DNA sequence encoding a protein, e.g., VEGFR or VEGF; (b) any DNA sequence that encodes a protein, e.g., VEGFR or VEGF amino acid sequence, and/or; (c) any DNA sequence that hybridizes to the complement of the coding sequences of a protein. In certain embodiments, the term includes coding as well as noncoding regions, and preferably includes all sequences necessary for normal gene expression.

The term “target gene” (alternatively referred to as “target gene sequence” or “target DNA sequence”) refers to any nucleic acid molecule, polynucleotide, or gene to be modified by homologous recombination. The target sequence includes an intact gene, an exon or intron, a regulatory sequence or any region between genes. The target gene may comprise a portion of a particular gene or genetic locus in the individual's genomic DNA.

The term “gene targeting” refers to a type of homologous recombination that occurs when a fragment of genomic DNA is introduced into a mammalian cell and that fragment locates and recombines with endogenous homologous sequences. Gene targeting by homologous recombination employs recombinant DNA technologies to replace specific genomic sequences with exogenous DNA of particular design.

The term “homologous recombination” refers to the exchange of DNA fragments between two DNA molecules or chromatids at the site of homologous nucleotide sequences.

“Disruption” of a gene, e.g., VEGFR or VEGF gene, occurs when a fragment of genomic DNA locates and recombines with an endogenous homologous sequence wherein the disruption is a deletion of the native gene or a portion thereof, or a mutation in the native gene or wherein the disruption is the functional inactivation of the native gene. Alternatively, sequence disruptions may be generated by nonspecific insertional inactivation using a gene trap vector (i.e. non-human transgenic animals containing and expressing a randomly inserted transgene; see for example U.S. Pat. No. 6,436,707 issued Aug. 20, 2002). These sequence disruptions or modifications may include insertions, missense, frameshift, deletion, or substitutions, or replacements of DNA sequence, or any combination thereof. Insertions include the insertion of entire genes, which may be of animal, plant, fungal, insect, prokaryotic, or viral origin. Disruption, for example, can alter the normal gene product by inhibiting its production partially or completely or by enhancing the normal gene product's activity. In one embodiment, the disruption is a conditional null disruption, wherein there is no significant expression of the VEGFR or VEGF gene upon the additional of an inducer.

As used herein, the term “transgene” refers to a nucleic acid sequence that is partly or entirely heterologous, i.e., foreign, to the transgenic animal into which it is introduced, or is homologous to an endogenous gene of the transgenic animal into which it is introduced, but which is designed to be inserted, or is inserted, into the animal's genome in such a way as to alter the genome of the cell into which it is inserted (e.g., it is inserted at a location that differs from that of the natural gene). A transgene can be operably linked to one or more transcriptional regulatory sequences and any other nucleic acid, such as introns, that may be necessary for optimal expression of a selected nucleic acid.

The term “native expression” refers to the expression of the full-length polypeptide encoded by a gene, e.g., a VEGFR or VEGF gene, at expression levels present in the wild-type mouse or in an equivalent conditional transgenic non-human animal control. Thus, a disruption in which there is “no native expression” of the endogenous gene, e.g., a VEGFR or VEGF gene, refers to a partial or complete reduction of the expression of at least a portion of a polypeptide encoded by an endogenous gene, e.g., an endogenous VEGFR or VEGF gene, of a single cell, selected cells, or all of the cells of a mammal.

The term “conditional knockout” refers to the conditional disruption of a VEGFR or VEGF gene wherein the conditional disruption results in: the functional inactivation of the native gene; the deletion of the native gene or a portion thereof; or a mutation in the native gene at a trigger of a certain event, e.g., by an inducer.

The term “conditional” refers to a dependence of some activity or event on a stimulus or signal or inducer.

The term “construct” refers to an artificially assembled DNA segment to be transferred into a target tissue, cell line or animal. Typically, the construct will include a gene or a nucleic acid sequence of particular interest, a marker gene and appropriate control sequences. As provided herein, a targeting VEGFR construct includes a DNA sequence homologous to at least one portion of a VEGFR gene and is capable of producing a disruption in a VEGFR gene in a host cell. As provided herein, a targeting VEGF construct includes a DNA sequence homologous to at least one portion of a VEGF gene and is capable of producing a disruption in a VEGF gene in a host cell.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

“Transformation” refers to introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described by Cohen, (1972) Proc. Natl. Acad. Sci. (USA), 69: 2110; and, and, Mandel et al. (1970) J. Mol. Biol., 53: 154, is generally used for prokaryotes or other cells that contain substantial cell-wall barriers. For mammalian cells without such cell walls, the calcium phosphate precipitation method of Graham and van der Eb, (1978) Virology, 52: 456-457, is used. General aspects of mammalian cell host system transformations have been described by Axel in U.S. Pat. No. 4,399,216 issued Aug. 16, 1983. Transformations into yeast are typically carried out according to the method of Van Solingen et al. (1977) J. Bact., 130: 946; and, Hsiao et al. (1979) Proc. Natl. Acad. Sci. (USA), 76: 3829. Other methods for introducing DNA into cells such as by nuclear injection or by protoplast fusion may also be used.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “transgenic cell” refers to a cell containing within its genome at least a VEGFR and/or VEGF gene that has been disrupted, modified, altered, or replaced completely or partially by the method of gene targeting.

The term “transgenic animal” refers to an animal that contains within its genome a specific gene that has been disrupted or otherwise modified or mutated by the methods described herein or methods otherwise well known in the art. In certain embodiments, the non-human transgenic animal is a mammal. In one embodiment, the mammal is a rodent such as a rat or mouse, a rabbit, a monkey, a guinea pig, a dog, a sheep, a horse, a dog, a cow, a cat, etc. In addition, a “transgenic animal” may be a heterozygous animal (i.e., one defective allele and one wild-type allele) or a homozygous animal (i.e., two defective alleles). An embryo is considered to fall within the definition of an animal. The provision of an animal includes the provision of an embryo or foetus in utero, whether by mating or otherwise, and whether or not the embryo goes to term.

The term “conditional transgenic non-human animal” or “non-human binary transgenic animal” refers to a non-human animal that has reduced expression of desired gene, in which gene expression is conditional, e.g., is controlled by the interaction of inducer. These interactions are controlled by crossing animal lines or by adding or removing an exogenous inducer. Such controlled gene expression alters the phenotype of the host cell compared to a host cell from an uncrossed line or lacking or having an exogenous inducer.

As used herein, the term “marker” refers to a reporter, a positive selection marker, and a negative selection marker. A reporter refers to any molecule the expression of which in a cell produces a detectable signal, e.g., detectable signal, e.g., luminescence. Exemplary markers are disclosed in, e.g., Sambrook, J., et al., (2001) Molecular Cloning: A Lbaroratory Manual, 2^{nd} ed. (2001), Cold Spring Harbor Laboratory Press, Cold Spring Harbor, N.Y.; and, U.S. Pat. No. 5,464,764 and No. 5,625,048. Many procedures for selecting and detecting markers are known in the art. See, e.g., Joyner, A. L., (2000) Gene Targeting: A Practical Approach, 2^{nd} ed., Oxford University Press, New York, N.Y.

As used herein, the terms “selective marker” and “position selection marker” refer to a gene, the expression of which allows cells containing the gene to be identified, e.g., antibiotic resistance genes and detectable, e.g., fluorescent, molecules. In certain embodiments of the invention, the gene encoding a product enables only the cells that carry the gene to survive and/or grow under certain conditions. For example, plant and animal cells that express the introduced neomycin resistance (Neo^{r}) gene are resistant to the compound G418. Cells that do not carry the Neo^{r} gene marker are killed by G418. Other positive selection markers are known to, or are within the purview of, those of ordinary skill in the art. A “negative selectin marker” refers to a gene, the expression of which inhibits cells containing the gene to be identified, e.g., the HSV-tk gene.

An “inducer” refers to a signal, regulator or regulatory molecule, an effector protein product, etc., capable of altering expression of a disrupted gene. An inducer may be delivered to a cell by means available in the art, including, for example, electroporation, transfection, infection, transport by cell receptors, diffusion across a cell membrane, or microinjection. In addition, an inducer may be delivered by, e.g., causing its transport into the nucleus from the cytoplasm, another organelle, or the cell or nuclear membrane. In certain embodiments of the invention, an inducer includes, but is not limited to, e.g., a polynucleotide, a polypeptide, a small organic molecule, etc. In certain embodiments of the invention, an inducer may be provided in the final form or they may be expressed from vectors introduced into a target cell or animal. Inducers can also be provided by mating an animal of the invention with a transgenic animal capable of expressing the inducer.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

A “native sequence” polypeptide comprises a polypeptide having the same amino acid sequence as a polypeptide derived from nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

The term “modulates” or “modulation” as used herein refers to the decrease, inhibition, reduction, amelioration, increase or enhancement of a VEGFR or VEGF gene function, expression, activity, or alternatively a phenotype associated with VEGFR or VEGF gene.

The term “modulator” or, “agent” or, alternatively, “compound” as used herein refers broadly to any substance with identifiable molecular structure and physiochemical property. Non-limiting examples of agents capable of modulating VEGFR or VEGF activities include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like.

The term “VEGFR modulator” refers to a molecule that can activate, e.g., an agonist, its expression, or that can inhibit, e.g., an antagonist (or inhibitor), the activity of a VEGFR or its expression. The term “VEGF modulator” refers to a molecule that can activate, e.g., an agonist (In this case the term “agonist” is defined in the context of the biological role of a VEGFR receptor), its expression, or that can inhibit, e.g., an antagonist (or inhibitor), the activity of a VEGF or its expression. The term “agonist” is used to refer to modulators above, e.g., peptide and non-peptide analogs of ligands of VEGFR, antibodies specifically binding such VEGFR, etc., provided they have the ability to signal through a native VEGFR receptor. An VEGFR antagonist refers to a molecule capable of neutralizing, blocking, inhibiting, abrogating, reducing or interfering with VEGFR activities, e.g., activities or phenotypes observed herein with the transgenic non-human mice, cell proliferation or growth, migration, adhesion or metabolic, including its binding to ligand, e.g., VEGF, VEGF selective variants, P1GF and VEGF-B. VEGFR antagonists include modulators described above, e.g., anti-VEGFR antibodies and antigen-binding fragments thereof, receptor molecules and derivatives which bind specifically to VEGFR thereby sequestering its binding to one or more ligands, anti-VEGFR ligand antibodies, anti-VEGF antibodies, and VEGFR antagonists such as small molecule inhibitors of the receptor. Other VEGFR antagonists also include antagonist variants of VEGFR, antisense molecules (e.g., VEGFR-SiRNA), RNA aptamers, and ribozymes against VEGFR or its receptor. In certain embodiments, antagonist VEGFR antibodies are antibodies that inhibit or reduce the activity of VEGFR by binding to a specific subsequence or region of VEGFR.

“Active” or “activity” for the purposes herein refers to form(s) of VEGFR or VEGF which retain a biological and/or an immunological activity of native or naturally-occurring VEGFR or VEGF, wherein “biological” activity refers to a biological function (either inhibitory or stimulatory) caused by a native or naturally-occurring VEGFR or VEGF other than the ability to induce the production of an antibody against an antigenic epitope possessed by a native or naturally-occurring VEGFR or VEGF and an “immunological” activity refers to the ability to induce the production of an antibody against an antigenic epitope possessed by a native or naturally-occurring VEGFR or VEGF.

The term “Anti-VEGFR antibody” is an antibody that binds to VEGFR with sufficient affinity and specificity. In certain embodiments of the invention, the anti-VEGFR antibody can be used as a therapeutic agent in targeting and interfering with diseases or conditions wherein VEGFR activity is involved. Generally, an anti-VEGFR antibody will usually not bind to other VEGFR homologues.

The term “Anti-VEGF antibody” is an antibody that binds to VEGF with sufficient affinity and specificity. In certain embodiments of the invention, the anti-VEGF antibody can be used as a therapeutic agent in targeting and interfering with diseases or conditions wherein VEGF activity is involved. Generally, an anti-VEGF antibody will usually not bind to other VEGF homologues.

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments so long as they exhibit the desired biological activity.

An antibody having a “biological characteristic” of a designated antibody is one which possesses one or more of the biological characteristics of that antibody which distinguish it from other antibodies that bind to the same antigen. In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

The term “ameliorates” or “amelioration” as used herein refers to a decrease, reduction or elimination of a condition, disease, disorder, or phenotype, including an abnormality or symptom.

A “disorder” is any condition that would benefit from treatment with an agent of the invention. This includes chronic and acute disorders or diseases including those pathological conditions which predispose the subject to the disorder in question.

Transgenic Animals

Trangenic animals are important models for disease. Transgenic knockout animals can be highly informative in the discovery of gene function and pharmaceutical utility for a drug target, as well as in the determination of the potential on-target side effects associated with a given target. For example, gene function and physiology are so well conserved between mice and humans, since they are both mammals and contain similar numbers of genes, which are highly conserved between the species. It has recently been well documented, for example, that 98% of genes on mouse chromosome 16 have a human ortholog (Mural et al., Science 296:1661-71 (2002)). Gene targeting in embryonic stem (ES) cells of mice has enabled the construction of mice with null mutations in many genes associated with human disease. One can also design valuable mouse models of human diseases by establishing a method for gene replacement (knock-in) which will disrupt the mouse locus and introduce a human counterpart with mutation, subsequently one can conduct in vivo drug studies targeting the human protein (Kitamoto et. Al., Biochemical and Biophysical Res. Commun., 222:742-47 (1996)).

However, not all genetic diseases are attributable to null mutations. Some transgenic knockouts are limited in the information they provide due to the early embryonic lethality of knocking out the gene, e.g., VEGF, VEGFR-1 or VEGFR-2 null mice result in an early embryonic lethality. See, e.g., Carmeliet, P. et al., Abnormal Blood Vessel Development and Lethality in Embryos Lacking a Single VEGF Allele, Nature 380:435-439 (1996); Shalaby, F., et al., Failure of Blood-Island Formation and Vasculogenesis in Flk-1 Deficient Mice, Nature 376:62-66 (1995); Fong, G. H., et al., Role of the Flt-1 receptor tyrosine kinase in regulating the assembly of vascular endothelium, Nature 376:66-70 (1995); and, Fong, G. H., Increased Hemangioblast Commitment, not vascular disorganization, is the primary defect in Flt-1 knock-out mice, Development. 126:3015-3025 (1999). Thus, the ability to study, the role of these receptors during early postnatal life and in the adult is still needed.

The invention provides conditional knockout cells and non-human animals (or binary transgenic cells and non-human animals) of a VEGFR and/or VEGF gene, which may be used to analyze the function of the VEGFR and/or VEGF gene, identify if VEGFR and/or VEGF is involved in disease, and to generate cell and animal models of diseases. In addition, the conditional knockouts may be used to identify the role of a VEGFR and/or VEGF at different stages of development or in different tissues. The invention also provides methods of using the conditional knockout cells and animals to identify agents or compounds, including, for example, small molecules, antibodies, etc., and methods of screening compounds to identify new drugs or new pharmaceutical indications for known drugs that modulate the activity of VEGFR and/or VEGF. The invention also provides high throughput screening assays to identify and select for agents or compounds that effect the activity of a VEGFR and/or VEGF gene product. In addition, the invention may be used to model the effects of drug administration, including acute administration, in a cell or animal.

In certain embodiments, a transgenic or knock out animal is a mammal, e.g., a rodent, a mouse, a rat, a rabbit, a monkey, a guinea pig, a dog, a sheep, a horse, a dog, a cow, a cat, etc. In certain embodiments of the invention, nucleic acids that encode a VEGFR or VEGF from non-human animal species, e.g., a rodent, a mouse, a rat, a rabbit, a monkey, a guinea pig, a dog, a sheep, a horse, a dog, a cow, a cat, etc., can be used to generate non-human transgenic or conditional (or binary) transgenic cells or animals, which, in turn, are useful in the development and screening of therapeutically useful agents or compounds. These nucleic acid sequences are readily available to one of skill in the art.

A transgenic animal is one having cells that contain a transgene, which was introduced into the animal or an ancestor of the animal, e.g., at a prenatal, such as, an embryonic stage. A transgene is a DNA that is integrated into the genome of a cell from which a transgenic animal develops. A conditional (or binary) transgenic animal is where there is conditional, i.e., temporal and spatial, control of gene expression in the animal. This can be achieved using binary transgenic systems, in which gene expression is controlled by the interaction of an inducer on a target transgene. These interactions are controlled by crossing animal lines (e.g., a rodent, a mouse, a rat, a rabbit, a monkey, a guinea pig, a dog, a sheep, a horse, a dog, a cow, a cat, etc.), or by adding or removing an exogenous inducer, as described in Lewandoski, (2001) Nature Reviews Genetics, 2: 743-755.

Binary transgenic systems fall into two categories. One is based on transcriptional transactivation and is well suited for activating transgenes in gain-of-function experiments. The other is based on site-specific DNA recombination and can be used to activate transgenes or to generate gene knockouts and cell-lineage markers. For example, conditional knockin/knockout technology includes, but is not limited to, e.g., Cre-LoxP, Flip-FLP recombinase, Tet-on/off technologies, etc. See, e.g., Lewandoski, (2001) Nature Reviews Genetics, 2: 743-755; U.S. Pat. Application No. 20040045043; Joyner, A. L. ed., Gene Targeting: A practical approach, 2^{nd} ed. (2000) Oxford University Press, New York; Kmiec, E. B. and Gruenert, D. C., eds., Gene Targeting Protocols (Methods in Molecular Biology, Vol. 133) (2000) Humana Press; and Torres, R. M. et al., (1997) Laboratory Protocols for Conditional Gene Targeting, Oxford University Press, Oxford.

Tet-on/off technology transcriptional system is based on the tetracycline resistance operon of E. coli. The effectors of these systems fall into two categories defined by whether transcription activation occurs upon the administration or depletion of a tetracycline compound (usually doxycycline). The Gal4-based system is a transactivation system that does not require an inducer, but Gal4 transcriptional activation can be controlled by synthetic steroids when a mutated ligand-binding domain is incorporated into a Gal4 chimeric transactivator.

A widely used site-specific DNA recombination system uses the Cre recombinase, e.g., from bacteriophage P1, or the Flp recombinase from S. cerevisiae, which has also been adapted for use in animals, e.g., mice. By using gene-targeting techniques to produce binary transgene animals with modified endogenous genes that can be acted on by Cre or Flp recombinases expressed under the control of tissue-specific promoters, site-specific recombination may be employed to inactivate endogenous genes in a spatially or time controlled manner. See, e.g., U.S. Pat. Nos. 6,080,576, 5,434,066, and 4,959,317; and Joyner, A. L., et al. Laboratory Protocols for Conditional Gene Targeting, Oxford University Press, New York (1997). The inducer, e.g., a recombinase, can be delivered a different stages. For example, a recombinase can be added to an embryonic stem cell containing a disrupted gene prior to the production of chimeras or implantation into an animal. In certain embodiments of the invention, the recombinase is delivered after the generation of an animal containing at least one disrupted gene allele. For example, the recombinase is delivered by cross breeding the animal containing a disrupted gene with an animal expressing the recombinase. The animal expressing the recombinase may express it, e.g., ubiquitously, in a tissue-restricted manner, or in a temporal-restricted manner.

Cre/Flp activity can also be controlled temporally by delivering cre/FLP-encoding transgenes in viral vectors, by administering exogenous steroids to the animals that carry a chimeric transgene consisting of the cre gene fused to a mutated ligand-binding domain, or by using transcriptional transactivation to control cre/FLP expression. In certain embodiments of the invention, mutated recombinase sites may be used.

In one embodiment, the transgenic animals are produced by introducing the VEGFR or VEGF transgene into the germine of the non-human animal. Methods for generating transgenic animals, particularly animals such as mice, are know in the art and are described, for example, in U.S. Pat. Nos. 4,736,866 and 4,870,009. See also, e.g., Lewandoski, (2001) Nature Review Genetics, 2:743-755; Raab, S., et al., Thromb. Haomost, 91:595-605 (2004); and, Haigh, J. J., et al., Developmental Biology, 262:225-241 (2003). In certain embodiments of the invention, particular cells would be targeted for transgene incorporation with tissue-specific enhancers. Embryonic target cells at various developmental stages can be used to introduce transgenes. Different methods are used depending on the stage of development of the embryonic target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used. The line(s) used to practice this invention may themselves be transgenic animals, and/or may be knockouts (i.e., obtained from animals that have one or more genes partially or completely suppressed).

The transgene construct may be introduced into a single-stage embryo. The zygote is the best target for microinjection. The use of zygotes as a target for gene transfer has a major advantage in that in most cases the injected DNA will be incorporated into the host gene before the first cleavage (Brinster et al., Proc. Natl. Acad. Sci. USA, 82: 44384442 (1985)). As a consequence, all cells of the transgenic animal will carry the incorporated transgene. This will in general also be reflected in the efficient transmission of the transgene to offspring of the founder, since 50% of the germ cells will harbor the transgene.

Normally, fertilized embryos are incubated in suitable media until the pronuclei appear. At about this time, the nucleotide sequence comprising the transgene is introduced into the female or male pronucleus. In some species such as mice, the male pronucleus is preferred. The exogenous genetic material may be added to the male DNA complement of the zygote prior to its being processed by the ovum nucleus or the zygote female pronucleus.

The exogenous genetic material may be added to the male complement of DNA or any other complement of DNA prior to its being affected by the female pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

In one embodiment of the invention, a conditional knockout animal of the invention is produced by introducing a vector construct of the invention into an embryonic stem cell line and selecting cells in which the introduced DNA has homologously recombined with the endogenous DNA (see e.g., Li et al., (1992) Cell, 69:915). The selected cells are then injected into a blastocyst of an animal (e.g., a mouse or rat) to form aggregation chimeras (see e.g., Bradley, in Teratocarcinomas and Embryonic Stem Cells: A Practical Approach, E. J. Robertson, ed. (IRL, Oxford, 1987), pp. 113-152). In a transgenic “knock out animal,” a chimeric embryo can then be implanted into a suitable pseudopregnant female foster animal. Progeny harboring the homologously recombined DNA in their germ cells can be identified by standard techniques and used to breed animals in which all cells of the animal contain the homologously recombined DNA. For example, a conditional knockout animal of the invention has a defective or altered gene encoding a VEGFR or VEGF protein (VEGFR or VEGF transgene) as a result of homologous recombination between the endogenous gene encoding VEGFR or VEGF and altered genomic DNA encoding VEGFR or VEGF introduced into an embryonic stem cell of the animal.

Non-human homologues of VEGFR or VEGF can be used to construct a VEGFR or VEGF, e.g., conditional, “knock out” animal. For example, cDNA encoding a VEGFR or VEGF can be used to clone genomic DNA encoding a VEGFR or VEGF in accordance with established techniques. A portion of the genomic DNA encoding the VEGFR or VEGF can be deleted or replaced with another gene, such as a gene encoding a selectable marker which can be used to monitor integration.

A vector construct of the invention comprises the whole or a fragment of the genomic sequence of a VEGFR or VEGF gene and a selection marker, e.g., a positive selection marker. Several kilobases of unaltered flanking DNA (both at the 5′ and 3′ ends) can be included in the vector (see e.g., Thomas and Capecchi, (1987) Cell, 51:503 for a description of homologous recombination vectors). In one aspect of the invention, the genomic sequence of the VEGFR or VEGF gene comprises at least part of an exon of VEGFR or VEGF gene. In certain embodiments of the invention, the exon is framed by recognition sites for a recombinase. In one embodiment of the invention, at least exon 1 of a VEGFR gene is included in the vector. In another embodiment, the entire VEGFR gene is included in the vector. In one embodiment of the invention, at least exon 3 of a VEGF gene is included in the vector. In another embodiment, the entire VEGF gene is included in the vector.

A selection marker of the invention can include a positive selection marker, a negative selection marker or include both a positive and negative selection marker. Examples of positive selection marker include but are not limited to, e.g., a neomycin resistance gene (neo), a hygromycin resistance gene, etc. In one embodiment, the positive selection marker is a neomycin resistance gene. In certain embodiments of the invention, the genomic sequence further comprises a negative selection marker. Examples of negative selection markers include but are not limited to, e.g., a diphtheria toxin gene, an HSV-thymidine kinase gene (HSV-TK), etc.

The transgenic animals produced in accordance with this invention typically include exogenous genetic material, e.g., a DNA sequence that can result in the conditional suppression of a VEGFR or VEGF. In certain embodiments of the invention, this exogenous genetic material is introduced by a vector construct of the invention. The sequence will be attached operably to a transcriptional control element, e.g., promoter, which allows the expression of the transgene production in a specific type of cell or specific time. For example, a conditional transgenic non-human animal or cell of the invention comprises a VEGFR or VEGF gene, a sequence element which permits conditional inhibition of the VEGFR or VEGF gene by an inducer. Expression of the disrupted VEGFR or VEGF gene within a cell or animal of the invention is approximately normal in the absence of an active inducer capable of altering expression of the disrupted VEGFR or VEGF gene via the sequence element. Typically, an inducer does not significantly effect the expression of a non-disrupted VEGFR or VEGF gene in the target cell or animal.

In certain embodiments of the invention, the expression or activity of the inducer, itself, may be regulated by another molecule. In one embodiment, an expression vector capable of induced expression of the inducer is provided to a cell or animal containing a disrupted gene capable of being regulated by the inducer. Similarly, in animals containing a transgene capable of expressing the inducer, the transgene may be regulated in an inducible or tissue- or stage-specific manner, via elements, e.g., transcriptional enhancer, located within the transgene. In certain embodiments of the invention, cells will be maintained in an agent or compound to avoid repression of the disrupted gene, while in other embodiments, an agent or compound will be added to induce repression of a disrupted gene. It is contemplated that expression of the inducer may be regulated by a conditional promoter and the expression of the targeted gene is regulated by inducing or inhibiting the expression of inducer.

Typically, particular cells would be targeted for VEGFR and/or VEGF transgene incorporation with tissue-specific enhancers. See, e.g., Raab, S., et al., Thromb. Haomost, 91:595-605 (2004); and, Haigh, J. J., et al., Developmental Biology, 262:225-241 (2003).

A transgene, e.g., a disrupted gene, an altered genomic DNA, a nucleic acid encoding a recombinase, etc., may be introduced into a cell, into an embryo, or animal by any means available in the art. Any technique that allows for the addition of the exogenous genetic material into nucleic genetic material can be utilized so long as it is not destructive to the cell, nuclear membrane, or other existing cellular or genetic structures. Such techniques include, but are not limited to transfection, scrape-loading or infection with a vector expressing the transgene, pronuclear microinjection (U.S. Pat. Nos. 4,873,191, 4,736,866 and 4,870,009); retrovirus mediated gene transfer into germ lines (an der Putten, et al., Proc. Natl. Acad. Sci., USA, 82:6148-6152 (1985)); gene targeting in embryonic stem cells (Thompson, et al., Cell, 56:313-321 (1989)); nonspecific insertional inactivation using a gene trap vector (U.S. Pat. No. 6,436,707); electroporation of embryos (Lo, Mol. Cell. Biol., 3:1803-1814 (1983)); lipofection; and sperm-mediated gene transfer (Lavitrano, et al., Cell, 57:717-723 (1989)); etc. In certain embodiments, the recombinase is delivered transiently, although other embodiments contemplate constitutive delivery of the recombinase, for example, by integration of a polynucleotide that expresses the transgene into a cell or animal genome.

In one example, exogenous genetic material is inserted into the nucleic genetic material by microinjection. Microinjection of cells and cellular structures is known and is used in the art. In the mouse, the male pronucleus reaches the size of approximately 20 micrometers in diameter, which allows reproducible injection of, e.g., 1-2 pL of DNA solution. Following introduction of the transgene nucleotide sequence into the embryo, the embryo may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host.

The number of copies of the transgene constructs that are added to the zygote depends on the total amount of exogenous genetic material added and will be the amount that enables the genetic transformation to occur. Theoretically only one copy is required. Generally numerous copies are utilized, for example, 1,000-20,000 copies of the transgene construct, to ensure that one copy is functional.

Retroviral infection can also be used to introduce the transgene into a non-human animal. The developing non-human embryo can be cultured in vitro to the blastocyst stage. During this time, the blastomeres can be targets for retroviral infection (Jaenich, Proc. Natl. Acad. Sci. USA, 73:1260-1264 (1976)). Efficient infection of the blastomeres is obtained by enzymatic treatment to remove the zona pellucida (Manipulating the Mouse Embryo, Hogan, ed. (Cold Spring Harbor Laboratory Press, Cold Spring Harbor, N.Y., 1986)). The viral vector system used to introduce the transgene is typically a replication-defective retrovirus carrying the transgene (Jahner et al., Proc. Natl. Acad. Sci. USA, 82: 6972-6931 (1985); and, Van der Putten et al., Proc. Natl. Acad. Sci. USA, 82: 6148-6152 (1985)). Transfection is easily and efficiently obtained by culturing the blastomeres on a monolayer of virus-producing cells (Van der Putten et al., supra; and, Stewart et al., EMBO J., 6: 383-388 (1987)). Alternatively, infection can be performed at a later stage. Virus or virus-producing cells can be injected into the blastocoele (Jahner et al., Nature, 298: 623-628(1982)). Most of the founders will be mosaic for the transgene since incorporation occurs only in a subset of the cells that formed the transgenic non-human animal. Further, the founder may contain various retroviral insertions of the transgene at different positions in the genome that generally will segregate in the offspring. In addition, it is also possible to introduce transgenes into the germ line by intrauterine retroviral infection of the mid-gestation embryo (Jahner et al. (1982), supra).

Transgenic offspring of the surrogate host may be screened for the presence and/or expression of the transgene by any suitable method. Screening is often accomplished by Southern blot or Northern blot analysis, using a probe that is complementary to at least a portion of the transgene. Typically, DNA is prepared from tail tissue and analyzed by Southern analysis or PCR for the transgene. Alternatively, the tissues or cells believed to altered expression of the transgene are tested for the presence and expression of the transgene using Southern analysis or PCR, although any tissues or cell types may be used for this analysis. See, e.g., southern hybridization. (Southern J. Mol. Biol. 98:503-517 (1975)), northern hybridization (see, e.g., Freeman et al. Proc. Natl. Acad. Sci. USA 80:4094-4098 (1983)), restriction endonuclease mapping (Sambrook et al. (2001) Molecular Cloning, A Laboratory Manual, 3rd Ed. Cold Spring Harbor Laboratory Press, New York), RNase protection assays (Current Protocols in Molecular Biology, John Wiley and Sons, New York, 1997), DNA sequence analysis, and polymerase chain reaction amplification (PCR; U.S. Pat. Nos. 4,683,202, 4,683,195, and 4,889,818; Gyllenstein et al. Proc Natl. Acad. Sci. USA 85:7652-7657 (1988); Ochman et al. Genetics 120:621-623 (1988); and, Loh et al. Science 243:217-220 (1989). Other methods of amplification commonly known in the art can be employed. The stringency of the hybridization conditions for northern or Southern blot analysis can be manipulated to ensure detection of nucleic acids with the desired degree of relatedness to the specific probes used. The expression of gene in a cell or tissue sample can also be detected and quantified using in situ hybridization techniques according to, for example, Current Protocols in Molecular Biology, John Wiley and Sons, New York, 1997.

Protein levels can be detected by immunoassays using antibodies specific to protein. For example, western blot analysis using an antibody against the VEGFR or VEGF encoded by the transgene may be employed as an alternative or additional method for screening for the presence of the transgene product. Various immunoassays known in the art can be used, including but not limited to competitive and non-competitive assay systems using techniques such as radioimmunoassay, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels), western blot analysis, precipitation reactions, agglutination assays (e.g., gel agglutination assays, hernagglutination assays), complement fixation assays, immunofluorescence assays, protein A assays, immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Alternative or additional methods for evaluating the presence of the transgene include, without limitation, suitable biochemical assays such as enzyme and/or immunological assays, histological stains for particular protein, marker or enzyme activities, flow cytometric analysis, and the like. Analysis of the bodily fluids may also be useful to detect the presence of the transgene product in the bodily fluid, as well as to evaluate the effect of the transgene on an organ, e.g., kidney, such as evaluating the presence of protein in the urine.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

Uses of Transgenic Animals

Knockout animals can be characterized for instance, for their ability to defend against certain pathological conditions and for their development of pathological conditions due to absence or conditional absence of the gene encoding the VEGFR and/or VEGF. Conditional (or binary) transgenic non-human animals can be used as tester animals for agents thought to modulate VEGFR and/or VEGF at, e.g., a particular development stage, etc. An animal treated with the agent/drug and having a reduced incidence of the disease, compared to untreated animals bearing the conditional (or binary) or ordinary transgene, would indicate a potential therapeutic intervention for the disease. In accordance with one facet of this aspect, for example, a conditional transgenic non-human animals with conditional targeted mutation in a VEGFR and/or VEGF gene in cells can be used to screen candidate drugs (proteins, peptides, polypeptides, small molecules, etc.), that modulating VEGFR and/or VEGF activity.

For example, transgenic animals that include a copy of a transgene encoding a VEGFR and/or VEGF introduced into the germ line of the animal at an embryonic stage can be used to examine the effect of decreased expression of DNA encoding VEGFR and/or VEGF polypeptides when the conditional mutation in the VEGFR and/or VEGF expression is triggered. Such animals can be used as tester animals for test agents thought to confer protection from, for example, pathological conditions associated with its absence or underexpression. In accordance with this facet of the invention, an animal is treated with the agent and a reduced incidence of the pathological condition, compared to untreated animals bearing the not triggered transgene, would indicate a potential therapeutic intervention for the pathological condition.

In certain embodiments, the invention encompasses methods of screening compounds to identify those that mimic the VEGFR agonists or prevent the effect of the VEGFR antagonists, e.g., at particular developmental stages. VEGFR agonists would be especially valuable therapeutically in the inducing activities of VEGFR and in those instances where a negative phenotype is observed based on findings with the non-human conditional transgenic animal whose genome comprises a conditional disruption of the gene which encodes for the VEGFR. VEGFR antagonists that prevent the effects VEGFR signaling would be especially valuable therapeutically in preventing VEGFR activities and in those instances where a positive phenotype is observed based upon observations with the non-human conditional transgenic knockout animal. Screening assays for antagonist drug candidates are designed to identify compounds that bind or complex with a VEGFR, or otherwise interfere with the interaction of the encoded polypeptide with other cellular proteins, e.g., a VEGFR ligand (e.g., VEGF, P1GF, VEGF-B, VEGF-C, VEGF-D, etc.). In certain embodiments, the invention encompasses methods of screening compounds to identify those that mimic a VEGF ligand (agonists) or prevent the effect of a VEGF ligand (antagonists).

For example, the effect of a VEGFR or VEGF antagonist can be assessed by administering a VEGFR or VEGF antagonist to a wild-type mouse in order to mimic a known knockout or conditional phenotype. Thus, one would initially conditionally knockout the VEGFR and/or VEGF gene of interest and observe the resultant phenotype as a consequence of conditionally knocking out or disrupting the VEGFR and/or VEGF gene. Subsequently, one could then assess the effectiveness of a VEGFR and/or VEGF antagonist by administering a VEGFR and/or VEGF antagonist to a wild-type mouse. An effective antagonist would be expected to mimic the phenotypic effect that was initially observed in the conditional knockout animal.

Likewise, one could assess the effect of a VEGFR or VEGF agonist, by administering a VEGFR or VEGF agonist to a non-human transgenic mouse in order to ameliorate a known negative conditional knockout phenotype. One can initially conditionally knockout the VEGFR and/or VEGF gene of interest and observe the resultant phenotype as a consequence of conditionally knocking out or disrupting the VEGFR and/or VEGF gene. Subsequently, the effectiveness of an agonist to the VEGFR and/or VEGF is assessed by administering a VEGFR or VEGF agonist to a non-human transgenic mouse. An effective agonist would be expected to ameliorate the negative conditional phenotypic effect that was initially observed in the conditional knockout animal.

In another assay for modulators, mammalian cells or a membrane preparation expressing the receptor would be incubated with a labeled VEGF in the presence of the candidate agent or compound. The ability of the agent or compound to enhance or block this interaction could then be measured. Alternatively, another assay for modulators, mammalian cells expressing the ligand would be incubated with a labeled VEGFR in the presence of the candidate agent or compound. The ability of the agent or compound to enhance or block this interaction could then be measured.

Recombinant Methods

Recombinant materials and methods are described herein and are known in the art. These materials and/or methods can also be applicable for generating the transgenic non-human animal, cells and vector constructs described herein. For example, a nucleic acid encoding a polypeptide of the invention (e.g., a VEGFR, VEGF, a polypeptide VEGFR modulator, polypeptide VEGF modulator, or other proteins described herein) can be isolated and inserted into a replicable vector for further cloning (amplification of the DNA), or expression. DNA encoding the polypeptide is readily isolated and sequenced using conventional procedures. For example, a DNA encoding a VEGFR or VEGF is isolated and sequenced, e.g., by using oligonucleotide probes that are capable of binding specifically to genes encoding the VEGFR or VEGF. Many vectors are available. The vector components generally include, but are not limited to, one or more of the following: a signal sequence, an origin of replication, one or more marker genes, an enhancer element, a promoter, and a transcription termination sequence.

Signal Sequence Component

Polypeptides of the invention may be produced recombinantly not only directly, but also as a fusion polypeptide with a heterologous polypeptide, which is typically a signal sequence or other polypeptide having a specific cleavage site at the N-terminus of the mature protein or polypeptide. The heterologous signal sequence selected typically is one that is recognized and processed (i.e., cleaved by a signal peptidase) by the host cell. For prokaryotic host cells that do not recognize and process the native polypeptide signal sequence, the signal sequence is substituted by a prokaryotic signal sequence selected, for example, from the group of the alkaline phosphatase, penicillinase, 1pp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the polypeptide of the invention.

Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Selection Gene Component

An expression vector, cloning vector or a vector construct of the invention may contain a selection gene, also termed a marker as described herein and below. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the nucleic acid, such as DHFR, thymidine kinase, metallothionein-I and -II, typically primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding a polypeptide of the invention, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid Yrp7 (Stinchcomb et al., (1979) Nature, 282:39). The trp1 gene provides a selection marker for a mutant strain of yeast lacking the ability to grow in tryptophan, for example, ATCC No. 44076 or PEP4-1. Jones, (1977) Genetics, 85:12. The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Promotor Component

Expression and cloning vectors and a vector construct of the invention usually contain a promoter that is recognized by the host organism and is operably linked to a nucleic acid encoding a polypeptide of the invention. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the polypeptide of the invention.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldyhyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldyhyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription of polypeptides of the invention from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and typically Simian Virus 40 (SV40), from heterologous mammalian promoters, e.g., the Flt1 promoter, the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the rous sarcoma virus long terminal repeat can be used as the promoter.

Enhancer Element Component

Transcription of a DNA encoding a polypeptide of this invention by higher eukaryotes is often increased by inserting an enhancer sequence into the vector. Many enhancer sequences are now known from mammalian genes (globin, elastase, albumin, α-fetoprotein, and insulin). Typically, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide-encoding sequence, but is typically located at a site 5′ from the promoter.

Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide of the invention. One useful transcription termination component is the bovine growth hormone polyadenylation region. See WO94/11026 and the expression vector disclosed therein.

Selection and Transformation of Host Cells

Suitable host cells for cloning or expressing DNA encoding the polypeptides of the invention in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Typically, the E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide of the invention-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptides of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70), African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for polypeptide of the invention production and cultured in conventional nutrient media modified as appropriate for inducing promoters, selecting transformants, or amplifying the genes encoding the desired sequences.

Culturing the Host Cells

The host cells used in the invention may be cultured in a variety of media. Commercially available media such as Ham's F10 (Sigma), Minimal Essential Medium ((MEM), (Sigma), RPMI-1640 (Sigma), Dulbecco's Modified Eagle's Medium ((DMEM), Sigma), etc., are suitable for culturing the host cells. In addition, any of the media described in Ham et al., (1979) Meth. Enz. 58:44, Barnes et al., (1980) Anal. Biochem. 102:255; U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with inducers, hormones and/or other growth factors (such as IFN-alpha, IFN-beta, insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Pharmaceutical Compositions

Therapeutic formulations of a VEGFR modulator or VEGF modulator are also provided and can be prepared by mixing a molecule, e.g., a polypeptide, having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Kit

In another embodiment of the invention, a kit containing a conditional transgenic non-human animal or cell with at least one disrupted VEGFR and/or VEGF gene is provided along with materials useful for the methods for screening for modulators of VEGFR and/or VEGF activity. In another embodiment of the invention, a kit containing a conditional transgenic non-human animal with both a disrupted VEGFR and VEGF gene is provided along with materials useful for the methods for screening for modulators of VEGFR and VEGF activity. Optionally, a set of instructions, generally written instructions, is included, which relates to the use and care of the conditional transgenic non-human animal with the disrupted VEGFR and/or VEGF gene. The instructions included with the kit generally include information to induce the conditional knockout of the VEGFR and/or VEGF resulting in no native expression.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

### Conditional Flt-1 and KDR Knockout Mice

To assess the role of VEGFR-1 and VEGFR-2 in postnatal development, the Cre-LoxP system was utilized to generate conditional knockouts of either receptor. To generate VEGFR-1-LOX and VEGFR-2-LOX mice, the gene-targeting strategy that is shown schematically in FIG. 2a was designed to delete the first coding exon and a region of the promotor immediately upstream of Exon1. In both cases, a targeting vector containing a PGK-neo cassette and three 34-base-pair LoxP sequences was electroporated into mouse embryonic stem cells. Following selection and identification of homologous recombinants, Cre-recombinase was transiently expressed to mediate LoxP recombination. Embryonic stem cell clones in which the PGK neo cassette was removed but two LoxP sites that flank exon 1 were maintained, were identified and confirmed by polymerase chain reaction and Southern blot analysis. See FIG. 2b. Two independently derived ES cell clones for the VEGFR-1-LOX and the VEGFR-2-LOX strategies were selected to generate separate mouse lines in each case. The insertion of these LoxP sequences upstream and downstream of exon 1 of the VEGFR-1 and VEGFR-2 genes did not disrupt the endogenous expression of these receptors as both strains of mice were born at the expected Mendelian frequency and did not display any phenotypic abnormalities.

VEGFR-1-LoxP and VEGFR-2-LoxP mice, in which the first coding exon and a region of the promotor of each gene is flanked by LoxP sites, were bred with Mx-1Cre mice in which the expression of Cre-recombinase is driven by an interferon (IFN)-α inducible gene promoter. See, e.g., Kuhn et al., Science 269:1427-1429 (1995). In these mice, Cre recombinase expression can be induced by alpha-interferon or by a form of double-stranded RNA known as pdIdC, that induces cells of the immune system to secrete interferon. Newborn VEGFR-1-LOX and VEGFR-2-LOX mice were injected with 10000 u or 20000 u of interferon-alpha on Day 1, Day 3 and Day 5 and monitored the growth and survival of these mice. See FIG. 3. Tissues and RNA was isolated from VEGFR-1 conditional knockouts and VEGFR-2 conditional knockouts and analysed by quantitative RT-PCR, and compared with the expression of the receptors in each case relative to the controls. In this Mx1Cre model, a reduction in VEGFR-1 expression was detected in the liver, kidney and thymus of VEGFR-1 conditional knockouts, and in VEGFR-2 conditional knockouts VEGFR-2 expression was reduced in the kidney. See FIG. 4. The expression of each receptor was elevated in the bone marrow of each of the respective conditional knockouts. See FIG. 4. In the resulting VEGFR-1-LoxP.Mx1-Cre and VEGFR-2-LoxP.Mx1-Cre mice, induction of Cre-recombinase expression with IFN-α (or the IFN-α inducer polyinosinic-polycytidylic acid) results in LoxP recombination with 95% efficiency in bone marrow, liver and spleen, and 50% or less efficiency in various organs, including kidney, heart and lung.

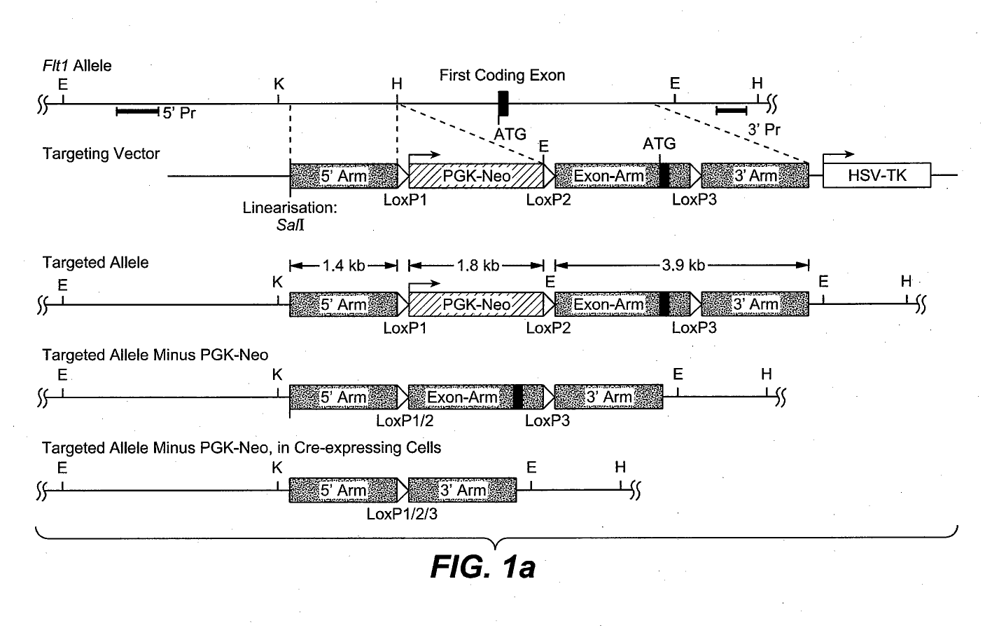

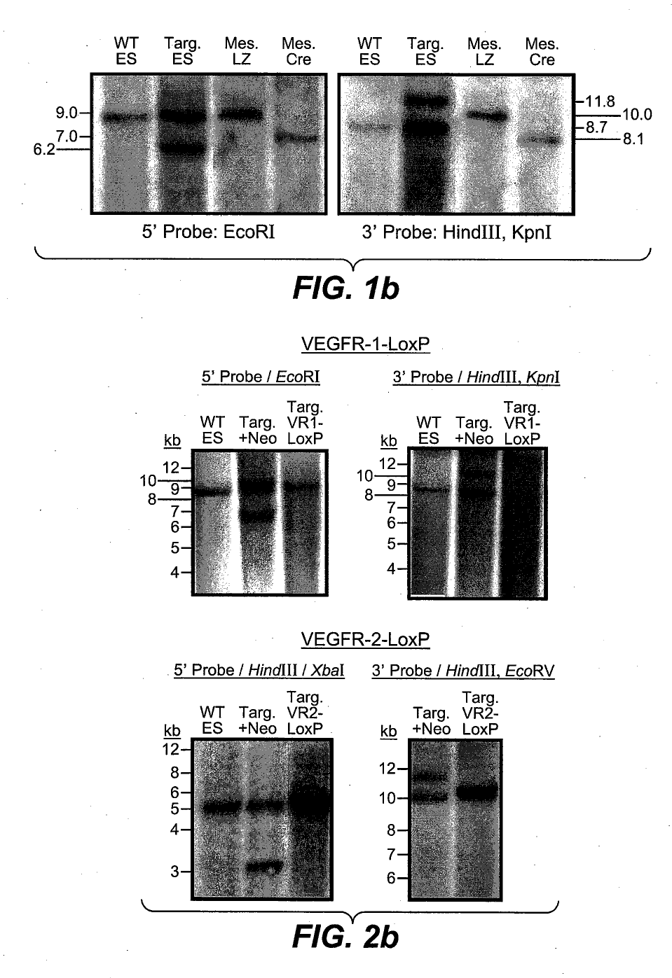

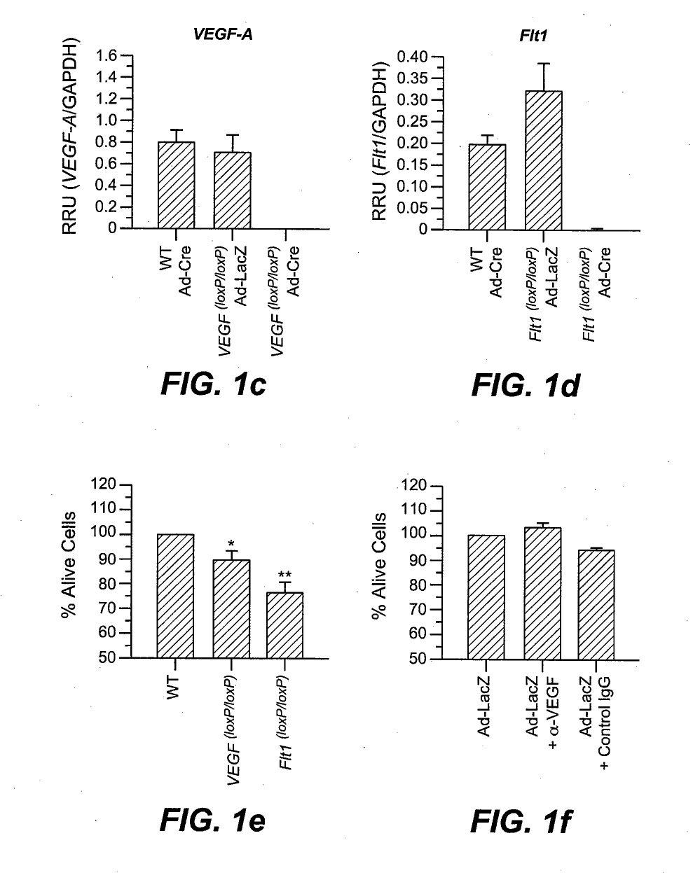

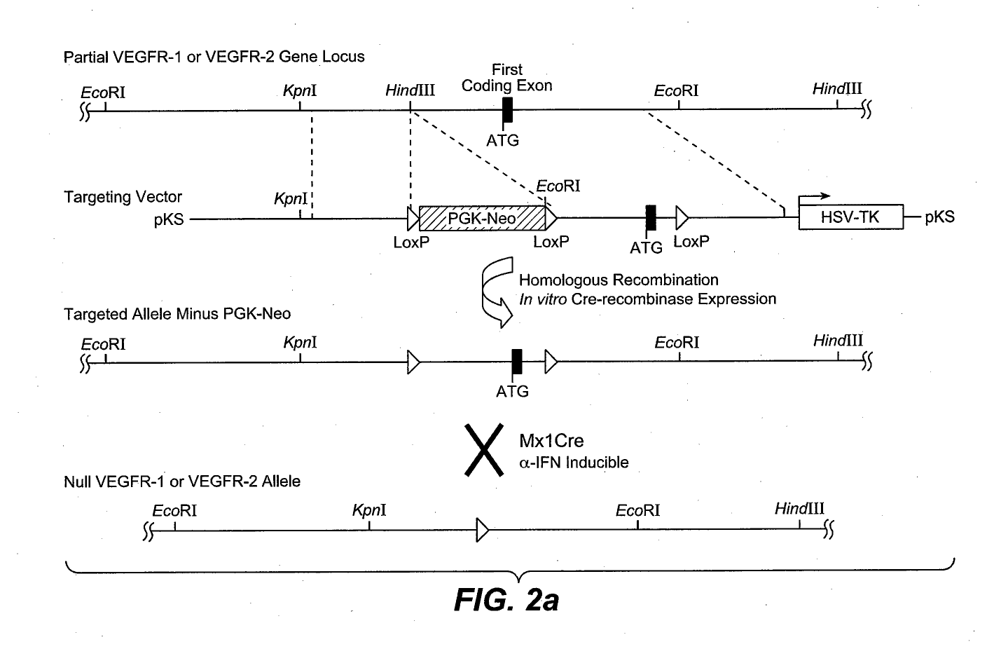

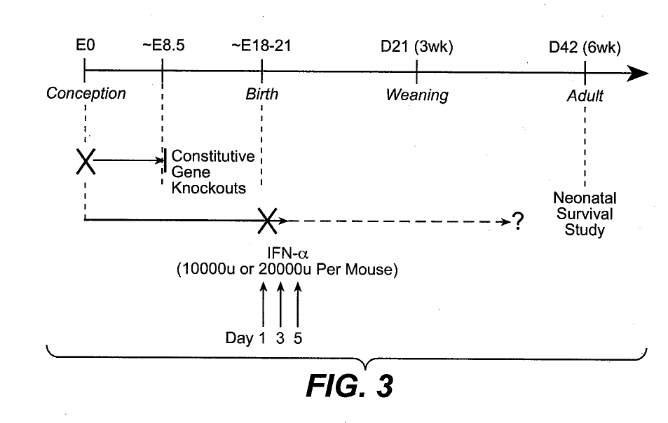

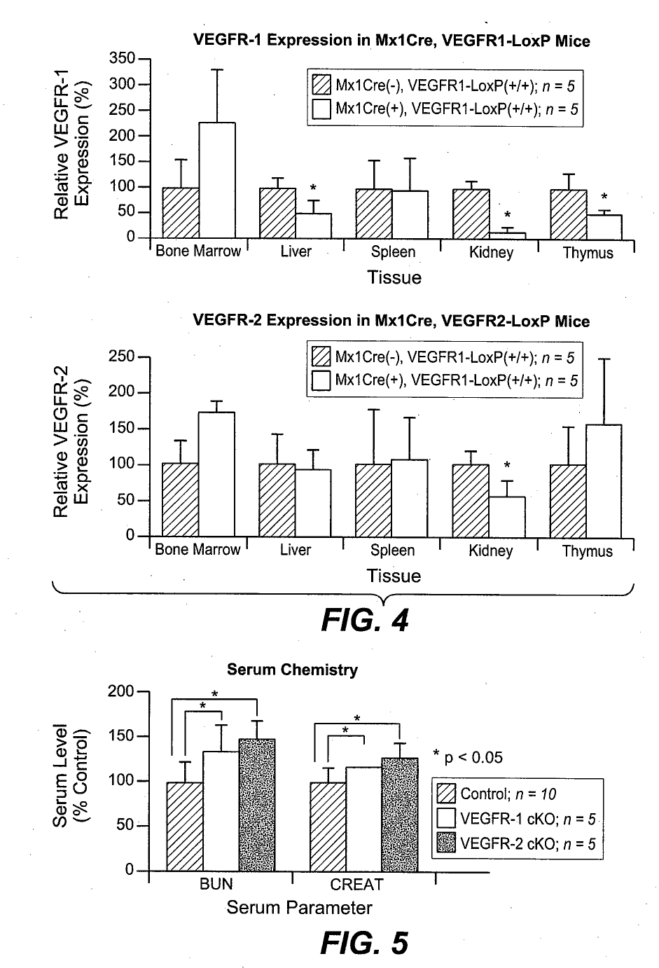

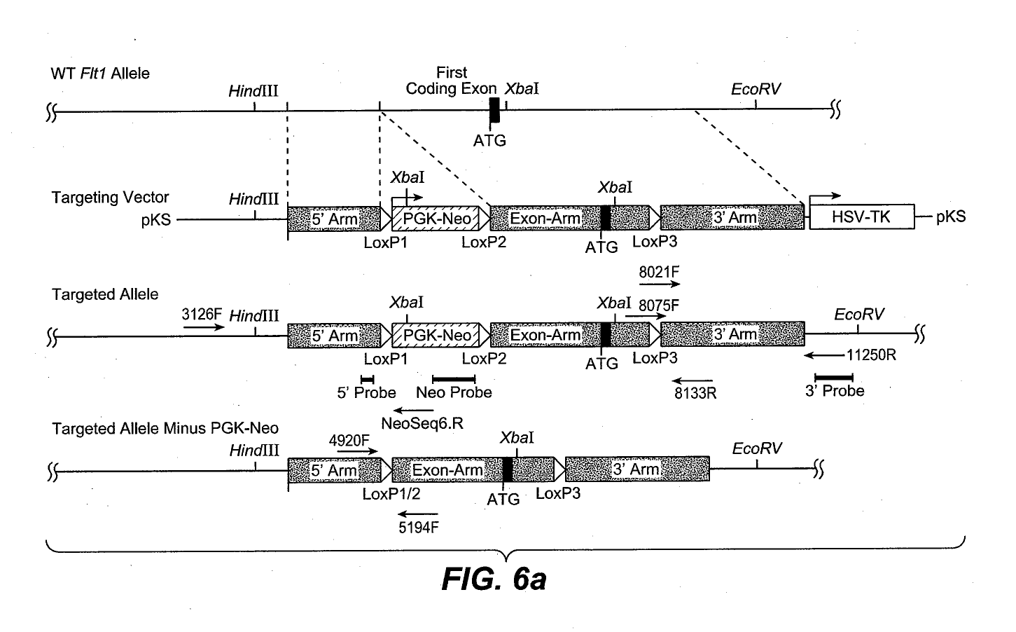

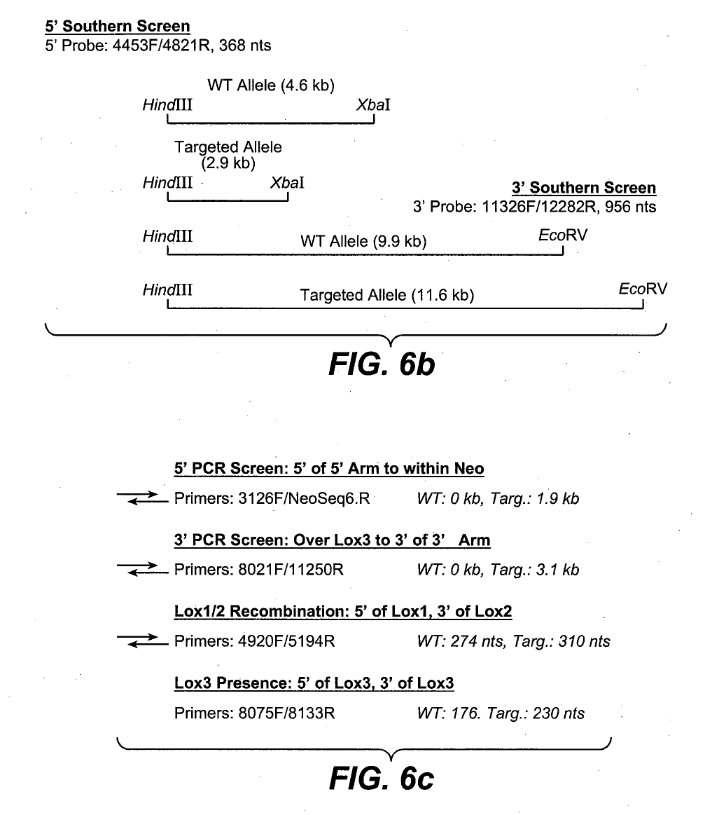

Generation of Flt1-loxP mice: A 16-kb genomic Flt-1 DNA clone encompassing exon 1 of the murine Flt1 gene locus was isolated following screening of a bacterial artificial chromosome library using the following primers: A 1.4 kb HindIII genomic DNA fragment spanning 3.0 to 1.6 kb upstream of the Flt1 translation initiation codon was excised and blunt-end cloned into the NotI site of TNLOX1-3 targeting vector. Subsequently, a 2.0 kb HindIII/BstXI genomic DNA fragment was cloned by blunt-end ligation into the unique AscI site of TNLOX1-3, downstream of the PGK-neo^{R} cassette and immediately 5′ of LoxP3, as indicated in FIG. 1a. This 2.0 kb fragment included a region of the Flt1 gene promotor, transcription start site and exon 1 of the Flt1 gene. Finally, a 2.0 kb BstXI/BsmI genomic DNA fragment was blunt-ended and cloned into the PmeI site immediately 3′ of the third loxP site, to generate the targeting vector denoted TKNeoFlt1-1. TKNeoFlt1-1 was sequenced and subjected to restriction endonuclease digestion to verify the sequence and orientation of the loxP sites and genomic DNA inserts.

The targeting vector was linearised by SalI digestion and 20 μg electroporated into TCL1 and R1 ES cells that are derived from the 129Sv strain. ES cells and mouse embryonic fibroblasts were maintained in culture in the presence of murine leukemia inhibitory factor (LIF) as previously described (see, e.g., Gerber, H. P., et al. (1999a). VEGF is required for growth and survival in neonatal mice. Development 126:1149-1159). ES cells were subjected to positive selection with G418 (400 μg/ml) 24 hours after electroporation and after nine days of this selection, individual colonies were picked, grown and screened for positive recombination events by Southern blot analysis. Genomic DNA from resistant clones was digested with either EcoRI (for analysis of the 5′ end of the targeting event) or with both HindIII and KpnI (for analysis at the 3′ end of the targeted genomic region). The probes used to screen the 5′ and 3′ ends of the targeted region were generated by PCR using the following primer pairs: 5′ Probe (639 nts): Flt-LOX.1123F (GAT GGC CTT GAG TAT ATC CTG (SEQ ID NO:1)) and Flt-LOX.1762R (CAG CTC TGG ACT CCA GCT TGC (SEQ ID NO:2)); 3′ Probe (834 nts): Flt-LOX.9733F (GGA AAC TAT GTG GCT GAT CTC (SEQ ID NO:3)) and Flt-LOX.10567R (GTG AGA GCC AAG ATC GAG GAG (SEQ ID NO: 4)). Two independent ES cell clones, designated #15 and #F7 were identified as homologous recombinants and transiently transfected with an expression vector encoding Cre-recombinase (pMC-Cre) as described previously (see, e.g., Gerber, H. P., et al. (1999a). VEGF is required for growth and survival in neonatal mice. Development 126:1149-1159). The transfected ES clones were picked to obtain individual colonies and screened by Southern blot and PCR for deletion of the PGK-neo^{R} cassette and recombination between LoxP1 and LoxP2. In addition to Southern blot analysis, the selected colonies were also analysed by PCR using the primers Flt-LOX.236F (TAG ACT CTG CGC GCC ATA ACT (SEQ ID NO: 5)) and Flt-LOX.2629R (CAC TAA GAA GGC AGA GGC CAA (SEQ ID NO:6)). Flt-LOX.236F anneals to DNA immediately 5′ and overlapping with the first 6 nucleotides of LoxP3, and used in combination with Flt-LOX.2629R (that is homologous to DNA downstream of the 3′ arm of homology). will only generate a PCR product from DNA containing LoxP3. These primers were used to further confirm that the third loxP site had not undergone recombination.

One ES cell clone derived from #15 and #F7, in which the PGK-neo^{R} cassette was removed (denoted #15.C1.H1 and #F7.A.E11 respectively), was injected into the blastocoele cavity of 3.5 day C57B1/6J blastocysts (see, e.g., Hogan et al., et al. (eds.). (1994) Manipulating the mouse embryo, (Cold Spring Harbor Laborator Press)). Chimeric males were mated with C57B1/6J female mice and the offspring screened for germline transmission by PCR analysis to detect LoxP1/2 and LoxP3. The PCR primers used to screen for the presence of LoxP1/2 were Flt-LOX.1335F (CCT GCA TGA TTC CTG ATT GGA (SEQ ID NO:7)) and Flt-LOX.3207R (GCC TAA GCT CAC CTG CGG (SEQ ID NO: 8)). The PCR primers used to screen for the presence of LoxP3 were Flt-LOX.236F and Flt-LOX.2629R. Flt1-LoxP(+/−) mice were then crossed to generate Flt1-LoxP(−/−) that do not carry a floxed Flt1 allele, Flt1-LoxP(+/−) which carry a single Flt1 allele that is floxed, and Flt1-LoxP(+/+) mice in which both alleles of Flt1 contain loxP sites. Flt1-loxP mice were typically genotyped by PCR using the Flt-LOX.1335F and Flt-LOX.3207R oligonucleotides.

Generation of Flk1-loxP P mice: A 12.9-kb genomic Flk-1 DNA clone encompassing exon 1 of the murine Flk1 gene locus was isolated following screening of a bacterial artificial chromosome library. A 1.8 kb genomic DNA fragment spanning 3.8 to 2.0 kb upstream of the Flk1 translation initiation codon was excised and blunt-end cloned into the NotI site of TNLOX1-3 targeting vector. Subsequently, a 3.2 kb genomic DNA fragment was cloned by blunt-end ligation into the unique AscI site of TNLOX1-3, downstream of the PGK-neo^{R} cassette and immediately 5′ of LoxP3, as indicated in FIG. 6a. This 3.2 kb fragment included a region of the Flk1 gene promotor (2.0 kb immediately 5′ to the translation initiation codon), exon 1 of the Flk1 gene and 1.2 kb immediately 3′ of exon 1. Finally, a 3.0 kb genomic DNA fragment (spanning 1.2 to 4.2 kb 3′ of exon 1) was blunt-ended and cloned into the targeting vector immediately 3′ of the third loxP site, to generate the targeting vector denoted TNLOX1-3/Flk (FIG. 6a). TNLOX1-3/Flk was sequenced and subjected to restriction endonuclease digestion to verify the sequence and orientation of the loxP sites and genomic DNA inserts.

The targeting vector was linearized and 20 μg electroporated into TCL1 and R1 ES cells that are derived from the 129Sv strain. ES cells and mouse embryonic fibroblasts were maintained in culture in the presence of murine leukemia inhibitory factor (LIF) as previously described (see, e.g., Gerber, H. P., et al. (1999a). VEGF is required for growth and survival in neonatal mice. Development 126:1149-1159). ES cells were subjected to positive selection with G418 (400 μg/ml) 24 hours after electroporation and after nine days of selection, individual colonies were picked, grown and screened for positive recombination events by Southern blot analysis. Genomic DNA from resistant clones was digested with both HindIII and XbaI (for analysis of the 5′ end of the targeting event) or with both HindIII and EcoRV (for analysis at the 3′ end of the targeted genomic region). The positions of the restriction endonuclease sites and the probes used to screen for homologous recombinants are indicated in FIG. 6b. The probes used to screen the 5′ and 3′ ends of the targeted region were generated by PCR using the following primer pairs: 5′ Probe (368 nts): Flk-LOX.4453F (GCC TTG GGG ATC CTC CTA TC (SEQ ID NO: 9)) and Flk-LOX.4821R (CGT GGT AAT CGC CCC ATT TAG (SEQ ID NO:10)); 3′ Probe (956 nts): Flk-LOX.11326F (GCT GGG AGT GTG GTC ATT CA (SEQ ID NO:11)) and Flk-LOX.12282R (CAG TGC CTT TCT GGA ACC TAT C (SEQ ID NO: 12)). Two independent ES cell clones, designated #B8 and #C2 were identified as homologous recombinants and transiently transfected with an expression vector encoding Cre-recombinase (pMC-Cre) as described previously (see, e.g., Gerber, H. P., et al. (1999a). VEGF is required for growth and survival in neonatal mice. Development 126:1149-1159). The transfected ES clones were picked to obtain individual colonies and screened by Southern blot and PCR for deletion of the PGK-neoR cassette and recombination between LoxP1 and LoxP2 (FIG. 6, b and c). In addition to Southern blot analysis, the selected colonies were also analyzed by PCR using the primers Flk-LOX.8021F (TAG AGA AGG CGC GCC ATA ACT (SEQ ID NO:13)) and Flk-LOX.11250R (GAA GCC CAG AAC ATC AAG CCA G (SEQ ID NO: 14)). Flk-LOX.8021F anneals to DNA immediately 5′ and overlapping with the first 6 nucleotides of LoxP3, and used in combination with Flt-LOX.11250R (that is homologous to DNA downstream of the 3′ arm of homology) will only generate a PCR product from DNA containing LoxP3. These primers were used to further confirm that the third loxP site had not undergone recombination.

One ES cell clone derived from each of #B8 and #C2, in which the PGK-neoR cassette was removed (denoted #B8.C11 and #C2.D12 respectively), was injected into the blastocoele cavity of 3.5 day C57B1/6J blastocysts (see, e.g., Hogan et al., et al. (eds.). (1994) Manipulating the mouse embryo, (Cold Spring Harbor Laborator Press)). Chimeric males were mated with C57B1/6J female mice and the offspring screened for germline transmission by PCR analysis to detect LoxP1/2 and LoxP3. The PCR primers used to screen for the presence of LoxP1/2 were Flk-LOX.4920F (GCC TTG TCT GGC CAC CGG GAT G (SEQ ID NO: 15)) and Flt-LOX.5194R (GGC TGC TTG GTG TAC CTA TCG (SEQ ID NO:16)). The PCR primers used to screen for the presence of LoxP3 were Flk-LOX.8075F (GAC TTG GTT CAT CAG GCT AG (SEQ ID NO:17)) and Flk-LOX.8133R (GAC GCT GTT AAG CTG CTA CAC (SEQ ID NO:18)). Flk1(loxP/+) mice were then crossed to generate WT mice that do not carry a floxed Flk1 allele, Flk1(loxP/+) mice which carry a single Flk1 allele that is floxed, and Flk1(loxP/loxP) mice in which both alleles of Flk1 contain loxP sites. Flk1-loxP mice were typically genotyped by PCR using the Flk-LOX.8075F and Flk-LOX.8133R oligonucleotides.

IFN-α administration (see FIG. 3) to newborn VEGFR-1-LoxP(+/+).Mx1-Cre(+) and VEGFR-2-LoxP(+/+).Mx1-Cre(+) mice adversely affected body mass gain and survival compared to wild-type controls, demonstrating a role for both receptors during early postnatal development. However, in these mice, short term genetic ablation of VEGFR-1 and VEGFR-2 in the liver, bone marrow and spleen, beginning at 6 weeks of age did not induce any significant changes in total body or organ mass.

There were some specific changes in the some of the organs. For example, hypocellularity and dilated vascular sinusoids in the bone marrow of conditional VEGFR-1 knockouts (VEGFR-1 cKO, α-IFN) was observed. Thymic lymphocyte depletion was associated with VEGFR1 and VEGFR2 conditional knockouts (VEGFR-1 cKO, α-IFN; VEGFR-2 cKO, α-IFN). There was elevated B.U.N. and serum creatinine in VEGFR1 and VEGFR2 conditional knockout mice. See FIG. 5.

VEGFR-1 and VEGFR-2 conditional gene knockouts displayed alterations in the kidney following α-IFN administration in neonates. There was also decreased glomerular vascularisation in VEGFR-2 conditional knockouts, e.g., as determined by (α-CD31).

### Example 2

### Generation of VEGF-loxP and Flt1-Cre Mice and Breeding to the ROSA26 Reporter Strain

Generation of VEGF-loxP and Flt1-Cre mice and breeding to the ROSA26 reporter strain: VEGF-loxP mice were generated as previously described (see, e.g., Gerber, H. P., et al. (1999a). VEGF is required for growth and survival in neonatal mice. Development 126:1149-1159). Briefly, in VEGF-loxP mice, exon 3 of VEGF is flanked by loxP sites, resulting in a null VEGF allele in cells that undergo loxP recombination. VEGF-loxP mice were bred with Flt1-Cre mice in which a 3.1 kb fragment of the Flt1 promotor (see, e.g., Gerber, H. P., et al. (1997). Differential transcriptional regulation of the two vascular endothelial growth factor receptor genes. Flt-1, but not Flk-1/KDR, is up-regulated by hypoxia. J Biol Chem 272:23659-23667) drives expression of Cre-recombinase. Flt1-Cre mice were generated by microinjecting a construct containing a 3.1 kb fragment of the Flt1 promotor, driving expression of Cre-recombinase, into mouse egg pronuclei as described previously. See, Hogan, B., et al. (eds.). Manipulating the mouse embryo, (Cold Spring Harbor Laborator Press, 1994). To monitor expression of Cre-recombinase, Flt-CRE⁺; VEGF-^{loxP/loxP)} or Flt-Cre+ mice were crossed to the ROSA26 reporter strain (see, e.g., Mao, X., et al. (1999). Improved reporter strain for monitoring Cre recombinase-mediated DNA excisions in mice. Proc Natl Acad Sci USA 96, 5037-5042), whereby the ubiquitous expression of β-galactosidase is inhibited by transcriptional/translational terminating signals. This ‘stopper-fragment’ is flanked by loxP sites and undergoes Cre-mediated excision resulting in expression of the β-galactosidase gene in cells that express Cre-recombinase.

Generation of Flt1-loxP mice: see above.

VEGF Gene Ablation In Vitro Adversely Affects Mesangial Cell Survival: Evidence of VEGF Acting Via an Internal Autocrine Loop in Mesangial Cells.

To investigate the effects of VEGF-A and Flt1 gene ablation on mesangial cell grown in vitro, we generated mice with conditional alleles for the Flt1 allele (Flt1-lox/loxP). See FIG. 1, Panel a. A. targeting vector in which exon 1 of the mouse Flt1 gene is flanked by loxP sites was generated and used for homologous recombination in mouse embryonic stem cells. Flt1(loxP/loxP) mice were born at the expected Mendelian frequencies, indicating that the presence of 2 loxP sites did not interfere with mouse development. Homogeneous preparations of mesangial cells from WT VEGF(loxP/loxP) and Flt1(loxP/loxP) mice were obtained from glomerular isolates and infected with either control adenovirus expressing LacZ (Ad-LacZ) or adenovirus expressing Cre-recombinase (Ad-Cre). Flt1 and VEGF-A gene ablation frequencies in vitro were monitored by Southern blot analysis (FIG. 1, Panel b) and real time RT-PCR (FIG. 1, Panels c and d) and found to be >95%. Flt-1 or VEGF-A gene ablation in mesangial cells caused a significant reduction in cell survival (FIG. 1, Panel e), indicating that VEGF regulates mesangial cell survival in a cell autonomous manner, mediated by Flt1. Addition of a neutralizing VEGF antibody (α-VEGF, G6-23) did not impact on mesangial cell survival (FIG. 1, Panel f). As G6-23 is excluded from the intracellular compartment, the failure to recapitulate the decrease in mesangial cell survival observed in VEGF-A- or Flt1-deficient mesangial cells indicates that VEGF-A may act via an internal autocrine loop. The reduction in survival of Flt1-deficient mesangial cells is greater than that caused by VEGF-A deficiency alone. Other ligands for Flt1, such as P1GF or VEGF-B, may also contribute.

The specification is considered to be sufficient to enable one skilled in the art to practice the invention. It is understood that the examples and embodiments described herein are for illustrative purposes only. Indeed, various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

We claim:

1. A transgenic non-human animal comprising a conditional targeted VEGFR-1 mutation, wherein the conditional targeted mutation comprises one or both alleles of a VEGFR-1 gene.

2. The transgenic non-human animal of claim 1, wherein the conditional targeted VEGFR-1 mutation comprises recognition sites for a recombinase framing a genomic sequence of VEGFR-1 encoding a polypeptide with VEGFR-1 activity, wherein the genomic sequence comprises at least one exon of a VEGFR-1 gene.

3. The transgenic non-human animal of claim 2, wherein the at least one exon comprises exon 1 of the VEGFR-1 gene.

4. The transgenic non-human animal of claim 1, wherein the non-human animal is selected from the group consisting of a rodent, a rat, a rabbit, a monkey, a guinea pig, a dog, a sheep, a horse, a dog and a cat.

5. The transgenic non-human animal of claim 1, wherein the non-human animal is a mouse.

6. The transgenic non-human animal of claim 1, wherein the transgenic non-human mouse expresses no native expression of VEGFR-1 compared to a control transgenic non-human animal.

7. A transgenic cell comprising a conditional targeted VEGFR-1 mutation, wherein the conditional targeted mutation comprises one or both alleles of a VEGFR-1 gene.

8. A vector construct comprising a genomic sequence of VEGFR-1 gene, wherein the genomic sequence comprises at least one exon of a VEGFR-1 gene and a positive selection marker and wherein the genomic sequence is framed by recognition sites for recombinase.

9. The vector construct of claim 8, wherein the at least one exon comprises exon 1 of the VEGFR-1 gene.

10. The vector construct of claim 8, wherein the positive selection marker is selected from the group consisting of a neomycin resistance gene and a hygromycin resistance gene.

11. The vector construct of claim 8, wherein the genomic sequence further comprises a negative selection marker.

12. The vector construct of claim 11, wherein the negative selection marker is selected from the group consisting of a diphtheria toxin gene and an HSV-thymidine kinase gene (HSV-TK).

13. A host cell comprising the vector construct of claim 8.

14. A method of producing a transgenic conditional targeted VEGFR-1 mutation non-human animal, the method comprising:
introducing a vector construct of claim 8 into an embryonic stem cell of a non-human animal; and
generating a heterozygous and/or homozygous transgenic animal from the embryonic stem cell, thereby producing the transgenic conditional targeted VEGFR-1 mutation non-human animal.

15. The method of claim 14, further comprises cross breeding the transgenic conditional targeted VEGFR-1 mutation non-human animal with second transgenic animal.

16. The method of claim 15, wherein the second transgenic animal is a transgenic non-human animal with an inducer.

17. The method of claim 15, wherein the second transgenic animal is a transgenic conditional VEGF targeted mutation non-human animal.

18. A method for determining VEGFR-1 function, the method comprising:
administering an inducer of conditional mutation of a VEGFR-1 gene to a conditional transgenic conditional targeted VEGFR-1 mutation non-human animal of claim 1, thereby producing an induced conditional transgenic non-human animal;
analysizing and comparing the induced conditional transgenic non-human animal with a non-induced conditional transgenic non-human animal; and,
determining differences, thereby determining the function of VEGFR-1.

19. A transgenic non-human animal comprising a conditional targeted VEGFR-2 mutation, wherein the conditional targeted mutation comprises one or both alleles of a VEGFR-2 gene.

20. The transgenic non-human animal of claim 19, wherein the conditional targeted VEGFR-2 mutation comprises recognition sites for a recombinase framing a genomic sequence of VEGFR-2 encoding a polypeptide with VEGFR-2 activity, wherein the genomic sequence comprises at least one exon of a VEGFR-2 gene.

21. The transgenic non-human animal of claim 20, wherein the at least one exon comprises exon 1 of the VEGFR-2 gene.

22. The transgenic non-human animal of claim 19, wherein the non-human animal is selected from the group consisting of a rodent, a rat, a rabbit, a monkey, a guinea pig, a dog, a sheep, a horse, a dog and a cat.

23. The transgenic non-human animal of claim 19, wherein the non-human animal is a mouse.

24. The transgenic non-human animal of claim 19, wherein the transgenic non-human animal expresses no native expression of VEGFR-2 compared to a control transgenic non-human animal.

25. A transgenic cell comprising a conditional targeted VEGFR-2 mutation, wherein the conditional targeted mutation comprises one or both alleles of a VEGFR-2 gene.

26. A vector construct comprising a genomic sequence of VEGFR-2 gene, wherein the genomic sequence comprises at least one exon of a VEGFR-2 gene and a positive selection marker and wherein the genomic sequence is framed by recognition sites for recombinase.

27. The vector construct of claim 26, wherein the at least one exon comprises exon 1 of the VEGFR-2 gene.

28. The vector construct of claim 26, wherein the positive selection marker is selected from the group consisting of a neomycin resistance gene and a hygromycin resistance gene.

29. The vector construct of claim 26, wherein the genomic sequence further comprises a negative selection marker.

30. The vector construct of claim 29, wherein the negative selection marker is selected from the group consisting of a diphtheria toxin gene and an HSV-thymidine kinase gene (HSV-TK).

31. A host cell comprising the vector construct of claim 26.

32. A method of producing a transgenic conditional targeted VEGFR-2 mutation non-human animal, the method comprising:
introducing a vector construct of claim 26 into an embryonic stem cell of a non-human animal; and
generating a heterozygous and/or homozygous transgenic animal from the embryonic stem cell, thereby producing the transgenic conditional targeted VEGFR-2 mutation non-human animal.

33. The method of claim 32, further comprises:
cross breeding the transgenic conditional targeted VEGFR-2 mutation non-human animal with second transgenic animal.

34. The method of claim 33, wherein the second transgenic animal is a transgenic non-human animal with an inducer.

35. The method of claim 33, wherein the second transgenic animal is a transgenic conditional VEGF targeted mutation non-human animal.

36. A method for determining VEGFR-2 function, the method comprising:
administering an inducer of a conditional mutation of a VEGFR-2 gene to a conditional transgenic conditional targeted VEGFR-2 mutation non-human animal of claim 19, thereby producing an induced conditional transgenic non-human animal;
analysizing and comparing the induced conditional transgenic non-human animal with a non-induced conditional transgenic non-human animal; and,
determining differences, thereby determining the function of VEGFR-2.

37. A transgenic non-human animal comprising a conditional targeted VEGFR mutation, wherein the conditional targeted mutation comprises one or both alleles of a VEGFR gene.

38. A transgenic non-human animal comprising a VEGFR-1-Cre transgenic non-human animal.

39. A transgenic non-human animal comprising a conditional VEGFR-1-loxP transgenic non-human animal.

40. A transgenic non-human animal comprising a conditional a VEGFR-2 loxP transgenic non-human animal.

41. A transgenic non-human animal comprising a VEGFR-1-Cre: VEGF-loxP transgenic non-human animal.
